(19) United States
(12) Patent Application Publication
(10) Pub. No.: US 2026/0306661 A1
(43) Pub. Date: Oct. 8, 2026

(54) CAPSIDS AND METHODS OF TARGETING NEURONS

(71) Applicants: Capsigen Inc., Vancouver, WA (US); Oregon Health & Science University, Portland, OR (US)

(72) Inventors: Hiroyuki NAKAI, West Linn, OR (US); Christina CHATZI, Portland, OR (US)

(21) Appl. No.: 19/479,936

(22) PCT Filed: Apr. 30, 2024
(86) PCT No.: PCT/US2024/027077

§ 371 Date: Oct. 29, 2025

Related U.S. Application Data
(60) Provisional application No. 63/499,457, filed on May 1, 2023.

(51) Int. Cl. A61K 48/00; C07K 14/005; C12N 15/86

(52) U.S. Cl. CPC A61K 48/0033; A61K 48/005; C07K 14/005; C12N 15/86; C12N 2750/14122; C12N 2750/14143

(57) ABSTRACT

Provided herein are AAV capsids and methods useful for transducing neurons, in particular, both upper and lower motor neurons. In some embodiments, the methods and compositions are useful for delivering a nucleic acid molecule to a neuron. In certain instances, a targeting sequence is used to direct capsids to neurons for the delivery of a nucleic acid molecule.

### CROSS-REFERENCE

This application claims the benefit of U.S. Provisional Application No. 63/499,457 filed on May 1, 2023, which is incorporated herein by reference.

### BACKGROUND

Motor neuron diseases (MNDs) are a group of neurological disorders that destroy motor neurons, the cells responsible for controlling skeletal muscle function for activities such as walking, breathing, speaking, and swallowing. Generally, messages or signals from upper motor neurons in the brain are typically transmitted to the brain stem, spinal cord, and then to muscles in the body via lower motor neurons. For example, upper motor neurons direct the lower motor neurons to produce muscle movements. When the muscles cannot receive signals from motor neurons, the muscles begin to weaken and shrink in size (e.g., muscle atrophy or wasting). The muscles may also exhibit to spontaneously twitching (fasciculations) and/or stiffness (spasticity) and overactive reflexes that make voluntary movements slow and difficult. Over time, individuals with MNDs may lose the ability to walk or control other movements.

### SUMMARY

Recombinant adeno-associated virus (AAV) vector-mediated gene therapy is one of the most promising approaches to ameliorate genetic forms of neuromuscular diseases. However, although AAV9 has been used clinically for the treatment of motor neuron disorders, the wild-type AAV9 still requires the use of high vector doses (>10¹⁴ vector genome (vg)/kg) to deliver a therapeutic effect. In addition, AAV9 transduces a wide range of cell types effectively, leading to substantial vector spillover to non-target cells in the body. Such a high dosage requirement results in a number of critical issues including off-target toxicities, a limited therapeutic window, and high cost of manufacturing.

The variant AAVs comprising the variant capsid proteins (e.g., variant capsids) described herein are useful for transducing motor neurons. For example, AAVs comprising the variant capsid proteins (e.g., variant capsids) described herein exhibit enhanced motor neuron tropism.

In some embodiments, provided herein are methods of delivering a nucleic acid molecule to a motor neuron in an individual, the method comprising: administering a variant adeno-associated virus (AAV) comprising the nucleic acid molecule to the individual, wherein the variant AAV comprises a variant capsid protein comprising an amino acid insertion within loop VIII, wherein the amino acid insertion comprises SEQ ID NO: 15.

In some embodiments, provided herein are methods of delivering a therapeutic nucleic acid molecule to a motor neuron in a subject having a motor neuron disease, the method comprising: administering a variant adeno-associated virus (AAV) comprising the therapeutic nucleic acid molecule to the individual, wherein the variant AAV comprises a variant capsid protein comprising an amino acid insertion within loop VIII, wherein the amino acid insertion comprises SEQ ID NO: 15.

In some embodiments, provided herein are methods of treating a motor neuron disease or disorder in an individual, the method comprising: administering a variant adeno-associated virus (AAV) comprising a therapeutic nucleic acid molecule to the individual, wherein the variant AAV comprises a variant capsid protein comprising an amino acid insertion within loop VIII, wherein the amino acid insertion comprises SEQ ID NO: 15. A motor neuron disorder generally refers to and encompasses a condition that disturbs the normal function of motor neurons. In certain embodiments, the motor neuron disorder is characterized by muscle atrophy or wasting and/or fasciculations.

In some embodiments, provided herein are methods of delivering a nucleic acid to a motor neuron, the method comprising: contacting the motor neuron with a variant adeno-associated virus (AAV) comprising the nucleic acid molecule, wherein the variant AAV comprises a variant capsid protein comprising an amino acid insertion within loop VIII, wherein the amino acid insertion comprises SEQ ID NO: 15.

In some embodiments, the motor neurons are upper motor neurons. In some embodiments, the motor neurons are lower motor neurons. In some embodiments, the motor neurons comprise both upper and lower motor neurons. In some embodiments, the motor neurons are in an individual. In some embodiments, the motor neurons are located within the frontal cortex in the brain. In some embodiments, the motor neurons are located within the spine. In some embodiments, the motor neurons are located within the ventral horn region of the spinal cord. In some embodiments, the motor neurons are located within the cervical region to the sacral region of the spine. In some embodiments, the motor neurons are located within the cervical region of the spine, the thoracic region of the spine, the lumbar region of the spine, or the sacral region of the spine.

In some embodiments, the variant capsid protein is a variant AAV serotype 9 (AAV9) capsid protein. In some embodiments, the variant capsid protein without the amino acid insertion comprises 85% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 95% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises SEQ ID NO: 9.

In some embodiments, the AAV9 capsid protein comprises an N272A mutation. In some embodiments, the variant capsid protein without the amino acid insertion comprises 85% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 95% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises SEQ ID NO: 14.

In some embodiments, the amino acid insertion further comprises a linker. In some embodiments, the amino acid insertion further comprises the formula:

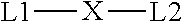

wherein:
L1 comprises a first amino acid linker sequence;
X comprises SEQ ID NO: 15; and
L2 is a second linker sequence.

In some embodiments, L1 and L2 are a different sequence. In some embodiments, L1 and L2 are an identical sequence.

In some embodiments, the variant capsid comprises SEQ ID NO: 15 and the remaining capsid sequence has at least 85%, 90%, 95%, 97%, 98%, 99%, or 100% sequence identity to SEQ ID NO: 16.

### BRIEF DESCRIPTION OF THE DRAWINGS

The novel features of the variant AAVs described herein are set forth with particularity in the appended claims. A better understanding of the features and advantages of the present variant AAVs described herein can be obtained by reference to the following detailed description that sets forth illustrative embodiments, in which the principles of the invention are utilized, and the accompanying drawings of which:

FIG. 1 shows a diagram of a TRAnscription-dependent Directed Evolution (TRADE) construct used for variant capsid discovery.

FIG. 2 shows data demonstrating that AAV-CAP2 exhibits widespread central nervous system (CNS) transduction in cynomolgus macaques. (A) shows quantification of neuronal transduction efficiency in different brain regions 3 weeks after the i.v. injection of AAV-CAP2-CAG-GFP. (B) shows representative confocal images of cynomolgus macaque brain sections immunostained with anti-GFP (green) and anti-NeuN (red) antibodies throughout various CNS regions of interest. Yellow/orange cells represent co-expression of GFP and NeuN. Scale bar, 100 μm. (C) shows quantification of neuronal specificity of AAV-CAP2-transduced cells in different brain regions. Four to six brain sections were stained and imaged, and technical replicates were pooled to obtain a single neuronal transduction efficiency or marker overlap for each region. Error bars are expressed as standard deviations.

FIG. 3 shows data demonstrating that AAV-CAP2 targets upper motor neurons/Betz cells in the cynomolgus macaque motor cortex. (A) shows representative images of DAB-GFP labeling in the primary motor cortex. (B) shows cell size distribution of GFP-positive transduced cells in the Layer Vb of the motor cortex of cynomolgus macaques. Frequency histogram depicting the number of motor neurons in each size bin (binned in 100-μm² steps) and the size of motor neurons (μm²). (C) shows representative image of double immunofluorescence microscopy against SMI-32 (red) and GFP (green). Scale bar, 50 μm. (D) shows quantification of upper motor neuron transduction efficiency. (E) Specificity of upper motor neurons among all the vector-transduced neurons. Four to 6 brain sections in the dorsal portion of Brodmann area 4 of the motor cortex were stained and imaged per animal and technical replicates were pooled to obtain the average for each animal. Data are presented as mean+/−standard deviations.

FIG. 4 shows data for AAV-CAP2-CAG-GFP vector genome biodistribution in the brain following systemic delivery in cynomolgus macaques. (A) shows vector genome copy number quantification in cortex and putamen samples by droplet digital PCR (ddPCR). (B) shows a table comparing results with vector genome copy numbers in the non-human primate (NHP) motor cortex reported in the literature.

FIG. 5 shows data demonstrating that intravenous AAV-CAP2 delivery mediates robust gene expression in the spinal cord. (A) shows a schematic representation of transverse (left) and longitudinal coronal (right) sections of the spinal cord. (B) shows representative DAB-GFP immunohistochemistry in coronal (B-D) and transverse sections (E-I) of the cervical (B,E,F), thoracic (C,G) and lumbar (D,H,I) segments of the spinal cord. DAB-GFP signal was detected in cells in the ventral horn with a motor neuron-like morphology. (E,I) show enlarged views of native GFP fluorescence signals in ventral horn cells in the cervical and lumbar segments (dotted lines), respectively.

FIG. 6 shows data demonstrating that AAV-CAP2 displays tropism for lower motor neurons after systemic delivery in cynomolgus macaques. (A-B) shows representative sections of the cervical (A) and lumbar (B) ventral horns from the cynomolgus macaque spinal cord observed by laser scanning confocal microscopy 21 days after the i.v. injection. Colocalization of ChAT-immunolabeled motor neurons (MN, red) with GFP (green) was observed in ventral horn. (C) shows quantification of lower motor neuron transduction in the cervical and lumbar segments. Data are presented as mean±/−standard deviations.

FIG. 7 shows data demonstrating thoracic and lumbar spinal cord tropism. (A) shows vector genome copy numbers per diploid genome detected in the spinal cord of 2 cynomolgus macaques 3 weeks after intravenous injection of AAV-CAP2-CAG-GFP. (B) shows a table comparing results with vector genome copy numbers in the NHP spinal cord reported in the literature.

FIG. 8. Shows data demonstrating DRG transduction with AAV-CAP2 in the cynomolgus macaques following i.v. injection. (A) shows a representative lumbar DRG sections stained 3 weeks after the i.v. injection of AAV-CAP2-CAG-GFP. Each section was immunostained with antibodies against GFP (green) and NeuN (red). Yellow/orange cells represent co-expression of GFP and NeuN. (B) shows quantifications of lumbar DRG sections expressing GFP. All data are presented as mean±/−standard deviations. (C) shows results of vector genome copy number assessments for lumbar DRGs of cynomolgus macaques.

FIG. 9 shows data showing biodistribution in peripheral organs. (A) Vector genome copy numbers per diploid genome in the liver and heart of 2 cynomolgus macaques, determined 3 weeks after intravenous injection of AAV-CAP2-CAG-GFP. (B) shows a table comparing results with vector genome copy numbers in NHP liver reported in the literature.

FIG. 10 shows data demonstrating AAV-CAG-CAP2-GFP neuronal transduction and specificity in the murine CNS. (A) shows quantification of GFP+ cells among the NeuN+ cells in motor cortex, striatum and thalamus. (B) shows quantification of total NeuN+ cells among the GFP+ cells in motor cortex, striatum and thalamus. N=5 mice per group. Data are presented as mean±/−standard deviations.

FIG. 11 shows data demonstrating AAV-CAP2 transduction in murine motor cortex. (A-B) show a representative image from murine primary motor cortex Layer Vb stained 3 weeks after the i.v. injection of AAV-CAP2-CAG-GFP. Yellow arrows: GFP+ cells with corticospinal upper motor neuron morphology. White arrows indicate GFP+ pyramidal cells with cortico-cortical morphology. Representative images of murine motor cortex immunostained with antibodies against GFP (green), NeuN (blue) and SMI-32 (red). Yellow arrows indicate SMI-32+/GFP+ cortico-spinal upper motor neurons. (C) shows quantification of upper motor neuron transduction in mice. (D) shows specificity of upper motor neurons among all the vector-transduced neurons (n=5 mice). Data are presented as mean+/−standard deviations.

FIG. 12 shows data demonstrating AAV-CAP2 transduction in murine spinal cord. (A) shows a representative section of the lumbar ventral horn from the murine spinal cord observed by laser scanning confocal microscopy 21 days after the i.v. injection. White arrows indicate colocalization of ChAT-immunolabeled motor neurons (MN) with GFP and NeuN observed in ventral horn. (B) shows quantification of pan-neuronal and motor neuron transduction efficiencies in the cervical and lumbar segments of the murine spinal cord (n=5 mice). Data are presented as mean±/−standard deviations.

FIG. 13 shows data demonstrating brain transduction with AAV-CAP2 in mice and cynomolgus macaques. (A) Shows neuronal transduction efficiency in the motor cortex, striatum and thalamus of mice (n=5) and cynomolgus macaques (n=2). (B) shows upper motor neuron transduction efficiencies in mice (n=5) and cynomolgus macaques (n=2). Data are presented as mean±/−standard deviations.

FIG. 14A-G show amino acid alignments of AAV serotypes.

### DETAILED DESCRIPTION

Provided herein are variant adeno-associated virus (AAV) capsids useful for targeting motor neurons. For example, the variant AAVs comprise variant capsids described useful for delivering a nucleic acid molecule to motor neurons. Generally, the variant capsids described herein utilize an amino acid insertion (e.g., SEQ ID NO: 15) that confers efficient and specific targeting (e.g., transduction) to motor neurons. The efficient and specific targeting of motor neurons achieved the variant capsids and their methods of use provide advantages over traditional wild-type capsid sequences (e.g., wild-type AAV9 of SEQ ID NO: 9) used for delivering a nucleic acid to motor neurons.

### Variant Capsids for Transducing Motor Neurons

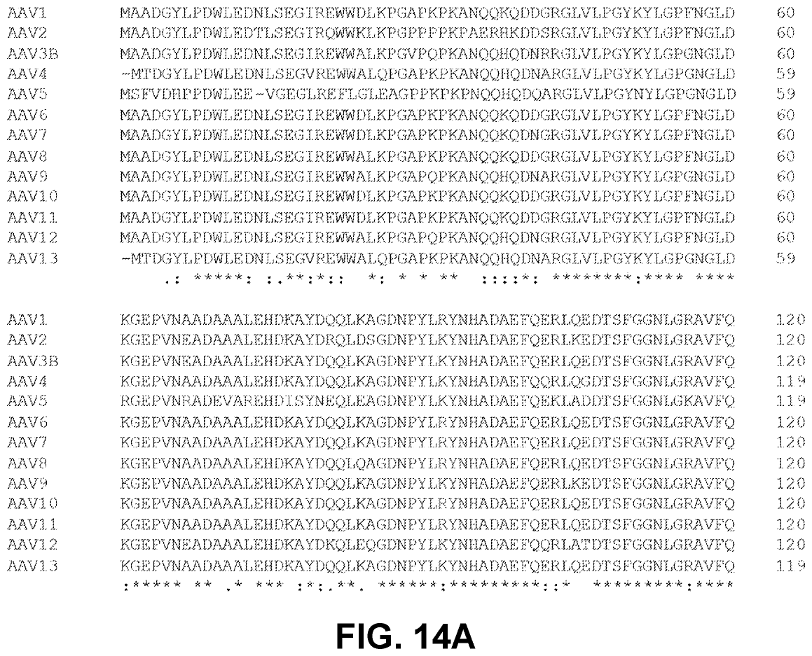

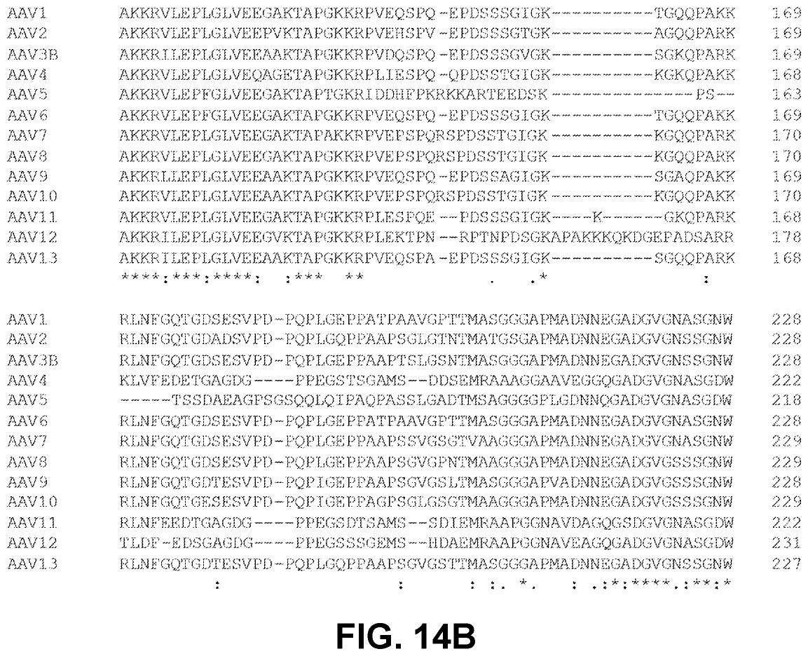

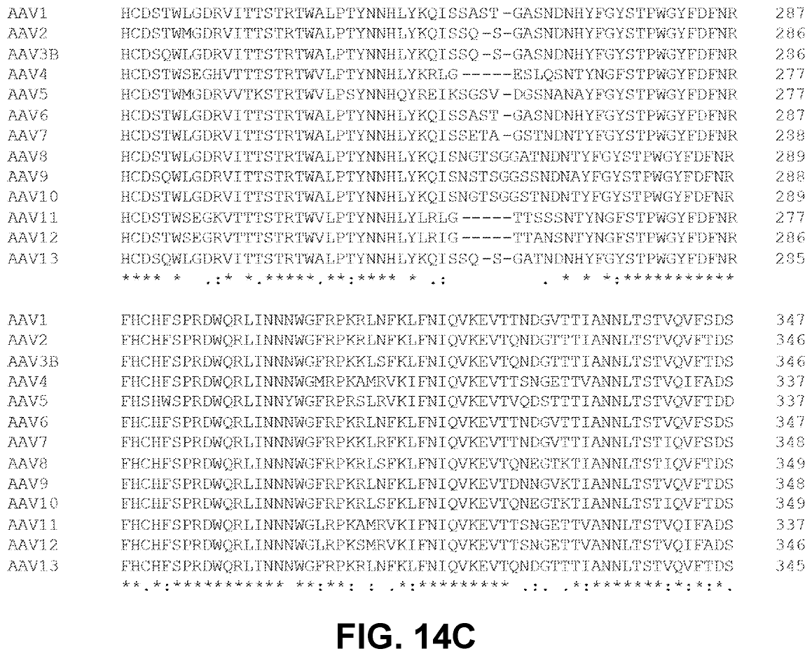

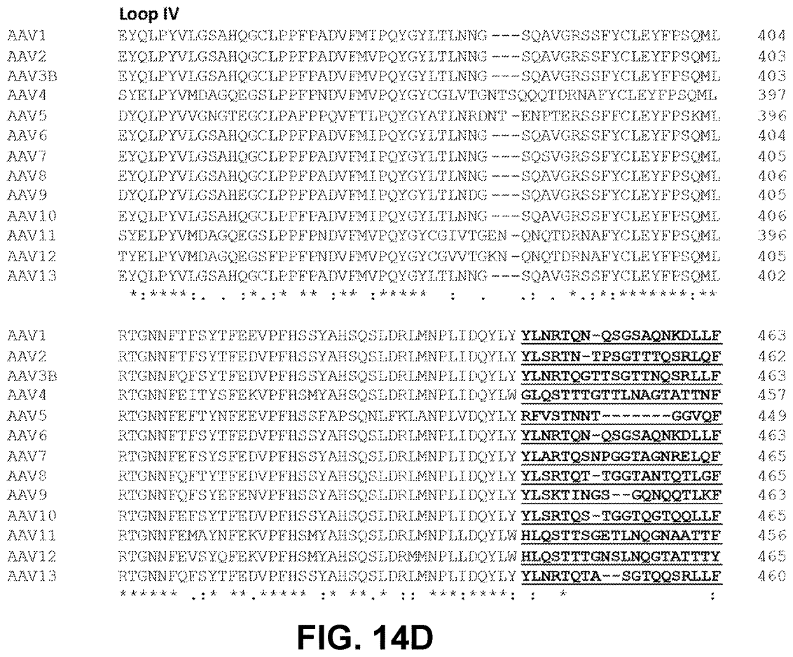

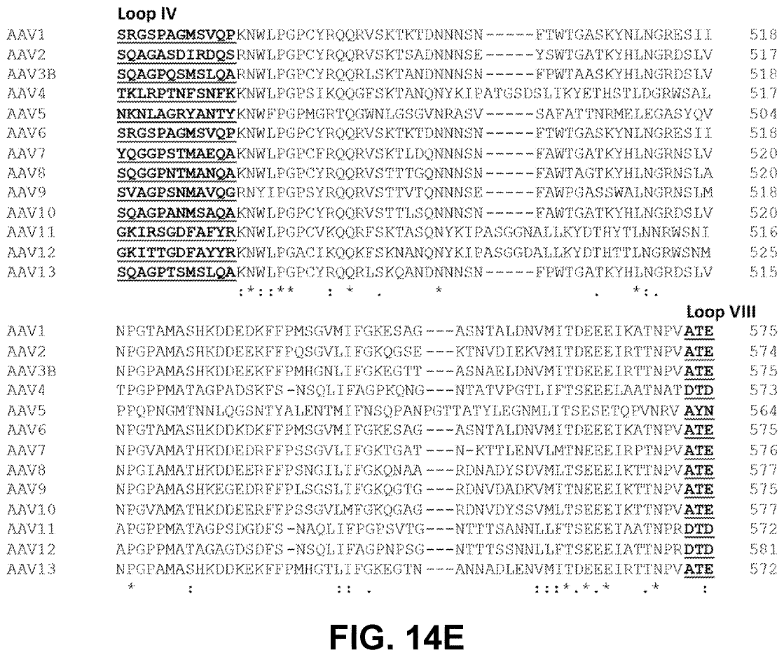

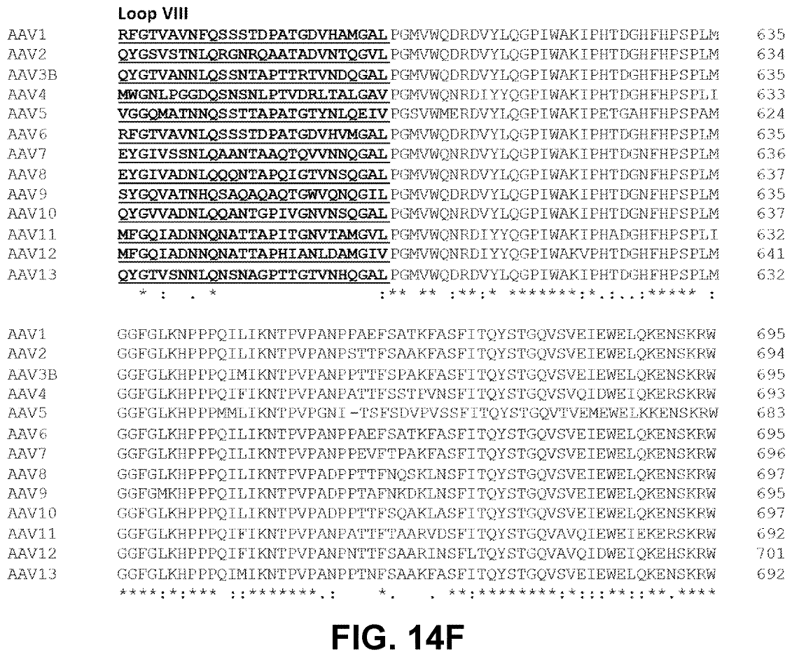

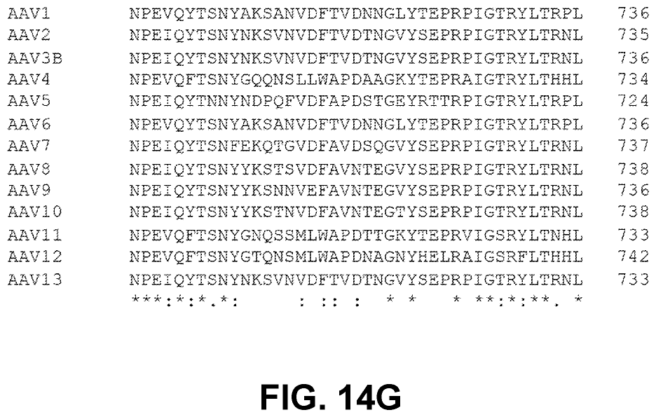

The variant AAVs comprising a variant capsid protein having an amino acid insertion comprising SEQ ID NO: 15 are use useful for transducing motor neurons. In some embodiments, the variant AAV comprises a variant capsid protein comprising an amino acid insertion within loop VIII, wherein the amino acid insertion comprises SEQ ID NO: 15. FIGS. 14E-F show an alignment of residues within loop VIII of an AAV. In some embodiments, loop VIII comprises the amino acid positions of Table 1.

TABLE 1

| | AAV | | | | | | | | | | | | |
|---|---|---|---|---|---|---|---|---|---|---|---|---|---|
| | 9 | 1 | 2 | 3B | 4 | 5 | 6 | 7 | 8 | 10 | 11 | 12 | 13 |
| Position | 575 | 575 | 574 | 575 | 573 | 564 | 575 | 576 | 577 | 577 | 572 | 581 | 572 |
| | 576 | 576 | 575 | 576 | 574 | 565 | 576 | 577 | 578 | 578 | 573 | 582 | 573 |
| | 577 | 577 | 576 | 577 | 575 | 566 | 577 | 578 | 579 | 579 | 574 | 583 | 574 |
| | 578 | 578 | 577 | 578 | 576 | 567 | 578 | 579 | 580 | 580 | 575 | 584 | 575 |
| | 579 | 579 | 578 | 579 | 577 | 568 | 579 | 580 | 581 | 581 | 576 | 585 | 576 |
| | 580 | 580 | 579 | 580 | 578 | 569 | 580 | 581 | 582 | 582 | 577 | 586 | 577 |
| | 581 | 581 | 580 | 581 | 579 | 570 | 581 | 582 | 583 | 583 | 578 | 587 | 578 |
| | 582 | 582 | 581 | 582 | 580 | 571 | 582 | 583 | 584 | 584 | 579 | 588 | 579 |
| | 583 | 583 | 582 | 583 | 581 | 572 | 583 | 584 | 585 | 585 | 580 | 589 | 580 |
| | 584 | 584 | 583 | 584 | 582 | 573 | 584 | 585 | 586 | 586 | 581 | 590 | 581 |
| | 585 | 585 | 584 | 585 | 583 | 574 | 585 | 586 | 587 | 587 | 582 | 591 | 582 |
| | 586 | 586 | 585 | 586 | 584 | 575 | 586 | 587 | 588 | 588 | 583 | 592 | 583 |
| | 587 | 587 | 586 | 587 | 585 | 576 | 587 | 588 | 589 | 589 | 584 | 593 | 584 |
| | 588 | 588 | 587 | 588 | 586 | 577 | 588 | 589 | 590 | 590 | 585 | 594 | 585 |
| | 589 | 589 | 588 | 589 | 587 | 578 | 589 | 590 | 591 | 591 | 586 | 595 | 586 |
| | 590 | 590 | 589 | 590 | 588 | 579 | 590 | 591 | 592 | 592 | 587 | 596 | 587 |
| | 591 | 591 | 590 | 591 | 589 | 580 | 591 | 592 | 593 | 593 | 588 | 597 | 588 |
| | 592 | 592 | 591 | 592 | 590 | 581 | 592 | 593 | 594 | 594 | 589 | 598 | 589 |
| | 593 | 593 | 592 | 593 | 591 | 582 | 593 | 594 | 595 | 595 | 590 | 599 | 590 |
| | 594 | 594 | 593 | 594 | 592 | 583 | 594 | 595 | 596 | 596 | 591 | 600 | 591 |
| | 595 | 595 | 594 | 595 | 593 | 584 | 595 | 596 | 597 | 597 | 592 | 601 | 592 |
| | 596 | 596 | 595 | 596 | 594 | 585 | 596 | 597 | 598 | 598 | 593 | 602 | 593 |
| | 597 | 597 | 596 | 597 | 595 | 586 | 597 | 598 | 599 | 599 | 594 | 603 | 594 |
| | 598 | 598 | 597 | 598 | 596 | 587 | 598 | 599 | 600 | 600 | 595 | 604 | 595 |
| | 599 | 599 | 598 | 599 | 597 | 588 | 599 | 600 | 601 | 601 | 596 | 605 | 596 |
| | 600 | 600 | 599 | 600 | 598 | 589 | 600 | 601 | 602 | 602 | 597 | 606 | 597 |
| | 601 | 601 | 600 | 601 | 599 | 590 | 601 | 602 | 603 | 603 | 598 | 607 | 598 |
| | 602 | 602 | 601 | 602 | 600 | 591 | 602 | 603 | 604 | 604 | 599 | 608 | 599 |
| | 603 | 603 | 602 | 603 | 601 | 592 | 603 | 604 | 605 | 605 | 600 | 609 | 600 |
| | 604 | 604 | 603 | 604 | 602 | 593 | 604 | 605 | 606 | 606 | 601 | 610 | 601 |
| | 605 | 605 | 604 | 605 | 603 | 594 | 605 | 606 | 607 | 607 | 602 | 611 | 602 |
| | 606 | 606 | 605 | 606 | 604 | 595 | 606 | 607 | 608 | 608 | 603 | 612 | 603 |
| | 607 | 607 | 606 | 607 | 605 | 596 | 607 | 608 | 609 | 609 | 604 | 613 | 604 |
| SEQ ID NO. | 9 | 1 | 2 | 3 | 4 | 5 | 6 | 7 | 8 | 10 | 11 | 12 | 13 |

In some embodiments, loop VIII comprises the amino acids corresponding to loop VIII of FIGS. 14E-F.

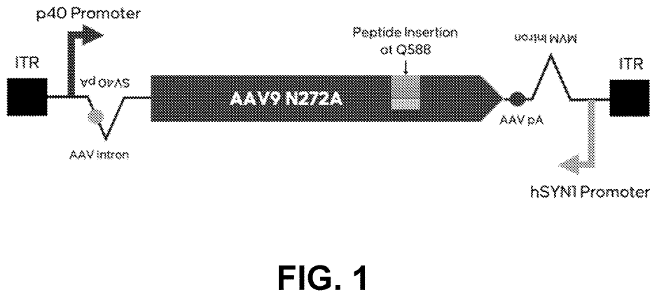

In some embodiments, the amino acid insertion comprising SEQ ID NO: 15 is inserted via substitution of an amino acid at the positions of Table 1. In some embodiments, the amino acid insertion comprising SEQ ID NO: 15 is inserted via substitution an amino acid within loop VIII of FIGS. 1E-F. In certain embodiments, the substitution insertion is further combined with a deletion of one or more amino acids within loop VIII. In some embodiments, the amino acid insertion comprising SEQ ID NO: 15 is inserted between two amino acids at the positions of Table 1. In some embodiments, the amino acid insertion comprising SEQ ID NO: 15 is inserted between two amino acids within loop VIII of FIGS. 1E-F. In certain embodiments, the insertion is between two nonconsecutive amino acids (e.g., 573 and 578), wherein intervening positions are deleted (e.g., 574-577). In some embodiments, the amino acid insertion comprises a substitution at position Q588 of SEQ ID NO: 9 or 14.

In some embodiments, the amino acid insertion comprising SEQ ID NO: 15 further comprises a linker. In certain embodiments, the amino acid insertion further comprises the formula:

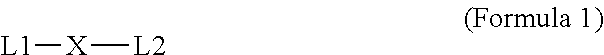

wherein:
L1 comprises a first amino acid linker sequence;
X comprises SEQ ID NO: 15; and
L2 is a second linker sequence.

In certain embodiments, the L1 and L2 are the same sequence. In certain embodiments, the L1 and L2 are the same sequence. In some embodiments, the amino acid linker comprises a glycine-serine linker, for example, (GS)n, (GSGGS)n, (GGGGS)n, and (GGGS)n, where n is an integer of at least one. In some embodiments, the amino acid linker comprises a glycine-alanine linker, alanine-serine linker, or other flexible linkers. In certain embodiments, the linker sequence comprises 1, 2, 3, 4, 5, 6, 7, 8, 9, 10, 12, 15, 20, 25 or greater amino acids.

In some embodiments, the variant capsid protein is a variant AAV serotype 9 (AAV9) capsid protein. In some embodiments, the AAV9 capsid protein comprises an N272A mutation.

In certain embodiments, the variant capsid protein without the amino acid insertion comprises 85% sequence identity to SEQ ID NO: 16. In certain embodiments, the variant capsid protein without the amino acid insertion comprises 90% sequence identity to SEQ ID NO: 9. In certain embodiments, the variant capsid protein without the amino acid insertion comprises 95% sequence identity to SEQ ID NO: 9. In certain embodiments, the variant capsid protein without the amino acid insertion comprises 96% sequence identity to SEQ ID NO: 9. In certain embodiments, the variant capsid protein without the amino acid insertion comprises 97% sequence identity to SEQ ID NO: 9. In certain embodiments, the variant capsid protein without the amino acid insertion comprises 98% sequence identity to SEQ ID NO: 9. In certain embodiments, the variant capsid protein without the amino acid insertion comprises 99% sequence identity to SEQ ID NO: 9. In certain embodiments, the variant capsid protein without the amino acid insertion comprises SEQ ID NO: 9.

In certain embodiments, the variant capsid protein without the amino acid insertion comprises 85% sequence identity to SEQ ID NO: 14. In certain embodiments, the variant capsid protein without the amino acid insertion comprises 90% sequence identity to SEQ ID NO: 14. In certain embodiments, the variant capsid protein without the amino acid insertion comprises 95% sequence identity to SEQ ID NO: 14. In certain embodiments, the variant capsid protein without the amino acid insertion comprises 96% sequence identity to SEQ ID NO: 14. In certain embodiments, the variant capsid protein without the amino acid insertion comprises 97% sequence identity to SEQ ID NO: 14. In certain embodiments, the variant capsid protein without the amino acid insertion comprises 98% sequence identity to SEQ ID NO: 14. In certain embodiments, the variant capsid protein without the amino acid insertion comprises 99% sequence identity to SEQ ID NO: 14. In certain embodiments, the variant capsid protein without the amino acid insertion comprises SEQ ID NO: 14.

In certain embodiments, the variant capsid protein without the amino acid insertion comprises 85% sequence identity to SEQ ID NO: 1. In certain embodiments, the variant capsid protein without the amino acid insertion comprises 90% sequence identity to SEQ ID NO: 1. In certain embodiments, the variant capsid protein without the amino acid insertion comprises 95% sequence identity to SEQ ID NO: 1. In certain embodiments, the variant capsid protein without the amino acid insertion comprises 96% sequence identity to SEQ ID NO: 1. In certain embodiments, the variant capsid protein without the amino acid insertion comprises 97% sequence identity to SEQ ID NO: 1. In certain embodiments, the variant capsid protein without the amino acid insertion comprises 98% sequence identity to SEQ ID NO: 1. In certain embodiments, the variant capsid protein without the amino acid insertion comprises 99% sequence identity to SEQ ID NO: 1. In certain embodiments, the variant capsid protein without the amino acid insertion comprises SEQ ID NO: 1.

In certain embodiments, the variant capsid protein without the amino acid insertion comprises 85% sequence identity to SEQ ID NO: 2. In certain embodiments, the variant capsid protein without the amino acid insertion comprises 90% sequence identity to SEQ ID NO: 2. In certain embodiments, the variant capsid protein without the amino acid insertion comprises 95% sequence identity to SEQ ID NO: 2. In certain embodiments, the variant capsid protein without the amino acid insertion comprises 96% sequence identity to SEQ ID NO: 2. In certain embodiments, the variant capsid protein without the amino acid insertion comprises 97% sequence identity to SEQ ID NO: 2. In certain embodiments, the variant capsid protein without the amino acid insertion comprises 98% sequence identity to SEQ ID NO: 2. In certain embodiments, the variant capsid protein without the amino acid insertion comprises 99% sequence identity to SEQ ID NO: 2. In certain embodiments, the variant capsid protein without the amino acid insertion comprises SEQ ID NO: 2.

In certain embodiments, the variant capsid protein without the amino acid insertion comprises 85% sequence identity to SEQ ID NO: 3. In certain embodiments, the variant capsid protein without the amino acid insertion comprises 90% sequence identity to SEQ ID NO: 3. In certain embodiments, the variant capsid protein without the amino acid insertion comprises 95% sequence identity to SEQ ID NO: 3. In certain embodiments, the variant capsid protein without the amino acid insertion comprises 96% sequence identity to SEQ ID NO: 3. In certain embodiments, the variant capsid protein without the amino acid insertion comprises 97% sequence identity to SEQ ID NO: 3. In certain embodiments, the variant capsid protein without the amino acid insertion comprises 98% sequence identity to SEQ ID NO: 3. In certain embodiments, the variant capsid protein without the amino acid insertion comprises 99% sequence identity to SEQ ID NO: 3. In certain embodiments, the variant capsid protein without the amino acid insertion comprises SEQ ID NO: 3.

In certain embodiments, the variant capsid protein without the amino acid insertion comprises 85% sequence identity to SEQ ID NO: 4. In certain embodiments, the variant capsid protein without the amino acid insertion comprises 90% sequence identity to SEQ ID NO: 4. In certain embodiments, the variant capsid protein without the amino acid insertion comprises 95% sequence identity to SEQ ID NO: 4. In certain embodiments, the variant capsid protein without the amino acid insertion comprises 96% sequence identity to SEQ ID NO: 4. In certain embodiments, the variant capsid protein without the amino acid insertion comprises 97% sequence identity to SEQ ID NO: 4. In certain embodiments, the variant capsid protein without the amino acid insertion comprises 98% sequence identity to SEQ ID NO: 4. In certain embodiments, the variant capsid protein without the amino acid insertion comprises 99% sequence identity to SEQ ID NO: 4. In certain embodiments, the variant capsid protein without the amino acid insertion comprises SEQ ID NO: 4.

In certain embodiments, the variant capsid protein without the amino acid insertion comprises 85% sequence identity to SEQ ID NO: 5. In certain embodiments, the variant capsid protein without the amino acid insertion comprises 90% sequence identity to SEQ ID NO: 5. In certain embodiments, the variant capsid protein without the amino acid insertion comprises 95% sequence identity to SEQ ID NO: 5. In certain embodiments, the variant capsid protein without the amino acid insertion comprises 96% sequence identity to SEQ ID NO: 5. In certain embodiments, the variant capsid protein without the amino acid insertion comprises 97% sequence identity to SEQ ID NO: 5. In certain embodiments, the variant capsid protein without the amino acid insertion comprises 98% sequence identity to SEQ ID NO: 5. In certain embodiments, the variant capsid protein without the amino acid insertion comprises 99% sequence identity to SEQ ID NO: 5. In certain embodiments, the variant capsid protein without the amino acid insertion comprises SEQ ID NO: 5.

In certain embodiments, the variant capsid protein without the amino acid insertion comprises 85% sequence identity to SEQ ID NO: 6. In certain embodiments, the variant capsid protein without the amino acid insertion comprises 90% sequence identity to SEQ ID NO: 6. In certain embodiments, the variant capsid protein without the amino acid insertion comprises 95% sequence identity to SEQ ID NO: 6. In certain embodiments, the variant capsid protein without the amino acid insertion comprises 96% sequence identity to SEQ ID NO: 6. In certain embodiments, the variant capsid protein without the amino acid insertion comprises 97% sequence identity to SEQ ID NO: 6. In certain embodiments, the variant capsid protein without the amino acid insertion comprises 98% sequence identity to SEQ ID NO: 6. In certain embodiments, the variant capsid protein without the amino acid insertion comprises 99% sequence identity to SEQ ID NO: 6. In certain embodiments, the variant capsid protein without the amino acid insertion comprises SEQ ID NO: 6.

In certain embodiments, the variant capsid protein without the amino acid insertion comprises 85% sequence identity to SEQ ID NO: 7. In certain embodiments, the variant capsid protein without the amino acid insertion comprises 90% sequence identity to SEQ ID NO: 7. In certain embodiments, the variant capsid protein without the amino acid insertion comprises 95% sequence identity to SEQ ID NO: 7. In certain embodiments, the variant capsid protein without the amino acid insertion comprises 96% sequence identity to SEQ ID NO: 7. In certain embodiments, the variant capsid protein without the amino acid insertion comprises 97% sequence identity to SEQ ID NO: 7. In certain embodiments, the variant capsid protein without the amino acid insertion comprises 98% sequence identity to SEQ ID NO: 7. In certain embodiments, the variant capsid protein without the amino acid insertion comprises 99% sequence identity to SEQ ID NO: 7. In certain embodiments, the variant capsid protein without the amino acid insertion comprises SEQ ID NO: 7.

In certain embodiments, the variant capsid protein without the amino acid insertion comprises 85% sequence identity to SEQ ID NO: 8. In certain embodiments, the variant capsid protein without the amino acid insertion comprises 90% sequence identity to SEQ ID NO: 8. In certain embodiments, the variant capsid protein without the amino acid insertion comprises 95% sequence identity to SEQ ID NO: 8. In certain embodiments, the variant capsid protein without the amino acid insertion comprises 96% sequence identity to SEQ ID NO: 8. In certain embodiments, the variant capsid protein without the amino acid insertion comprises 97% sequence identity to SEQ ID NO: 8. In certain embodiments, the variant capsid protein without the amino acid insertion comprises 98% sequence identity to SEQ ID NO: 8. In certain embodiments, the variant capsid protein without the amino acid insertion comprises 99% sequence identity to SEQ ID NO: 8. In certain embodiments, the variant capsid protein without the amino acid insertion comprises SEQ ID NO: 8.

In certain embodiments, the variant capsid protein without the amino acid insertion comprises 85% sequence identity to SEQ ID NO: 10. In certain embodiments, the variant capsid protein without the amino acid insertion comprises 90% sequence identity to SEQ ID NO: 10. In certain embodiments, the variant capsid protein without the amino acid insertion comprises 95% sequence identity to SEQ ID NO: 10. In certain embodiments, the variant capsid protein without the amino acid insertion comprises 96% sequence identity to SEQ ID NO: 10. In certain embodiments, the variant capsid protein without the amino acid insertion comprises 97% sequence identity to SEQ ID NO: 10. In certain embodiments, the variant capsid protein without the amino acid insertion comprises 98% sequence identity to SEQ ID NO: 10. In certain embodiments, the variant capsid protein without the amino acid insertion comprises 99% sequence identity to SEQ ID NO: 10. In certain embodiments, the variant capsid protein without the amino acid insertion comprises SEQ ID NO: 10.

In certain embodiments, the variant capsid protein without the amino acid insertion comprises 85% sequence identity to SEQ ID NO: 11. In certain embodiments, the variant capsid protein without the amino acid insertion comprises 90% sequence identity to SEQ ID NO: 11. In certain embodiments, the variant capsid protein without the amino acid insertion comprises 95% sequence identity to SEQ ID NO: 11. In certain embodiments, the variant capsid protein without the amino acid insertion comprises 96% sequence identity to SEQ ID NO: 11. In certain embodiments, the variant capsid protein without the amino acid insertion comprises 97% sequence identity to SEQ ID NO: 11. In certain embodiments, the variant capsid protein without the amino acid insertion comprises 98% sequence identity to SEQ ID NO: 11. In certain embodiments, the variant capsid protein without the amino acid insertion comprises 99% sequence identity to SEQ ID NO: 11. In certain embodiments, the variant capsid protein without the amino acid insertion comprises SEQ ID NO: 11.

In certain embodiments, the variant capsid protein without the amino acid insertion comprises 85% sequence identity to SEQ ID NO: 12. In certain embodiments, the variant capsid protein without the amino acid insertion comprises 90% sequence identity to SEQ ID NO: 12. In certain embodiments, the variant capsid protein without the amino acid insertion comprises 95% sequence identity to SEQ ID NO: 12. In certain embodiments, the variant capsid protein without the amino acid insertion comprises 96% sequence identity to SEQ ID NO: 12. In certain embodiments, the variant capsid protein without the amino acid insertion comprises 97% sequence identity to SEQ ID NO: 12. In certain embodiments, the variant capsid protein without the amino acid insertion comprises 98% sequence identity to SEQ ID NO: 12. In certain embodiments, the variant capsid protein without the amino acid insertion comprises 99% sequence identity to SEQ ID NO: 12. In certain embodiments, the variant capsid protein without the amino acid insertion comprises SEQ ID NO: 12.

In certain embodiments, the variant capsid protein without the amino acid insertion comprises 85% sequence identity to SEQ ID NO: 13. In certain embodiments, the variant capsid protein without the amino acid insertion comprises 90% sequence identity to SEQ ID NO: 13. In certain embodiments, the variant capsid protein without the amino acid insertion comprises 95% sequence identity to SEQ ID NO: 13. In certain embodiments, the variant capsid protein without the amino acid insertion comprises 96% sequence identity to SEQ ID NO: 13. In certain embodiments, the variant capsid protein without the amino acid insertion comprises 97% sequence identity to SEQ ID NO: 13. In certain embodiments, the variant capsid protein without the amino acid insertion comprises 98% sequence identity to SEQ ID NO: 13. In certain embodiments, the variant capsid protein without the amino acid insertion comprises 99% sequence identity to SEQ ID NO: 13. In certain embodiments, the variant capsid protein without the amino acid insertion comprises SEQ ID NO: 13.

In some embodiments, the variant capsid protein comprises 85% sequence identity to SEQ ID NO: 16. In some embodiments, the variant capsid protein comprises 90% sequence identity to SEQ ID NO: 16. In some embodiments, the variant capsid protein comprises 95% sequence identity to SEQ ID NO: 16. In some embodiments, the variant capsid protein comprises 96% sequence identity to SEQ ID NO: 16. In some embodiments, the variant capsid protein comprises 97% sequence identity to SEQ ID NO: 16. In some embodiments, the variant capsid protein comprises 98% sequence identity to SEQ ID NO: 16. In some embodiments, the variant capsid protein comprises 99% sequence identity to SEQ ID NO: 16. In some embodiments, the variant capsid protein comprises SEQ ID NO: 16.

In some embodiments, the variant capsid protein comprises SEQ ID NO: 15 and the remaining capsid sequence comprises at least 85% sequence identity to SEQ ID NO: 16. In some embodiments, the variant capsid protein comprises SEQ ID NO: 15 and the remaining capsid sequence comprises at least 90% sequence identity to SEQ ID NO: 16. In some embodiments, the variant capsid protein comprises SEQ ID NO: 15 and the remaining capsid sequence comprises at least 95% sequence identity to SEQ ID NO: 16. In some embodiments, the variant capsid protein comprises SEQ ID NO: 15 and the remaining capsid sequence comprises at least 96% sequence identity to SEQ ID NO: 16. In some embodiments, the variant capsid protein comprises SEQ ID NO: 15 and the remaining capsid sequence comprises at least 97% sequence identity to SEQ ID NO: 16. In some embodiments, the variant capsid protein comprises SEQ ID NO: 15 and the remaining capsid sequence comprises at least 98% sequence identity to SEQ ID NO: 16. In some embodiments the variant capsid protein comprises SEQ ID NO: 15 and the remaining capsid sequence comprises at least 99% sequence identity to SEQ ID NO: 16. In some embodiments, the variant capsid protein comprises SEQ ID NO: 16.

Percent sequence identity or percent identity or sequence identity generally refer to and encompass the number of identical matched positions shared between two polynucleotide or polypeptide sequences over a comparison window, taking into account additions or deletions (i.e., gaps) that may be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

In some embodiments, percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). BI2seq performs a comparison between two sequences using either the BLASTN or BLASTP algorithm. BLASTN is used to compare nucleic acid sequences, while BLASTP is used to compare amino acid sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See worldwideweb.ncbi.nlm.nih.gov. Other suitable programs are, e.g., Needle, Stretcher, ALIGN, Water, or Matcher, part of the EMBOSS suite of bioinformatics programs and also available from the European Bioinformatics Institute (EBI) at www.ebi.ac.uk/Tools/psa.

An adeno-associated virus or AAV or AAV vector generally refers to and encompasses an assembled viral capsid comprising a packaged nucleic acid molecule. An AAV capsid refers to an assembled viral capsid comprising a capsid protein. In some embodiments, an AAV capsid is a functional AAV capsid, e.g., is fully folded and/or assembled, is competent to infect a target cell (e.g., motor neuron), and/or is capable of remaining stable (e.g., folded/assembled and/or competent to infect a target cell). Wild-type AAV capsid protein sequences are encompassed by SEQ ID NOs: 1-13. In some embodiments, an AAV capsid protein comprises at least 70% or greater (e.g., 85%, 90%, 95%, or 100%) sequence identity to any one of SEQ ID NOs: 1-13. FIGS. 14A-G show alignment of AAV capsid protein sequences along with an annotation of select structural elements.

### Transduction of Motor Neurons

Motor neurons generally refer to and encompass a subset of neurons where the cell body is located in the motor cortex, brainstem, or the spinal cord and the axon projects to the spinal cord or outside the spinal cord and directly or indirectly controls muscles and glands. In some embodiments, motor neurons are choline acetyltransferase positive and/or SMI-32 positive. In certain embodiments, the motor neurons are also NeuN positive. In some embodiments, the motor neurons are upper motor neurons. In some embodiments, the motor neurons are lower motor neurons. In some embodiments, the motor neurons comprise both upper and lower motor neurons. In some embodiments, the motor neurons are in an individual. In some embodiments, the motor neurons are located within the spine. In some embodiments, the motor neurons are located within the ventral horn region of the spinal cord. In some embodiments, the motor neurons are located within the cervical region to the sacral region of the spine. In some embodiments, the motor neurons are located within the cervical region of the spine, the thoracic region of the spine, the lumbar region of the spine, or the sacral region of the spine.

In some embodiments, the variant AAV comprising a variant capsid protein comprising an amino acid insertion within loop VIII, wherein the amino acid insertion comprises SEQ ID NO: 15, exhibits a higher transduction (e.g., as measured by vector-mediated transgene expression or vector genomes per diploid genomic equivalent) of choline acetyltransferase-positive motor neurons when compared to transduction attainable by an equivalent dose of a control AAV (e.g., AAV9) comprising a wild-type AAV9 capsid protein (SEQ ID NO: 9). In some embodiments, the variant AAV exhibits reduced transduction of choline acetyltransferase-negative neurons and non-neuronal cells (e.g., glia) when compared to a control AAV (e.g., AAV9) comprising a wild-type AAV9 capsid protein (SEQ ID NO: 9).

In some embodiments, the variant AAV exhibits increased transduction (e.g., as measured by vector-mediated transgene expression or vector genomes per diploid genomic equivalent) of upper motor neurons when compared to transduction attainable by an equivalent dose of a control AAV (e.g., AAV9) comprising a wild-type AAV9 capsid protein (SEQ ID NO: 9). In some embodiments, the variant AAV exhibits reduced transduction of non-neuronal cells (e.g., glia), when compared to a control AAV (e.g., AAV9) comprising a wild-type AAV9 capsid protein (SEQ ID NO: 9).

In some embodiments, the variant AAV transduces (e.g., as measured by vector-mediated transgene expression or vector genomes per diploid genomic equivalent) a higher ratio of choline acetyltransferase-positive motor neurons to choline acetyltransferase-negative neurons and non-neuronal cells (e.g., glia) when compared to a control AAV (e.g., AAV9) comprising a wild-type AAV9 capsid protein (SEQ ID NO: 9). In some embodiments, the variant AAV transduces a higher ratio of upper motor neurons to other types of neurons and non-neuronal cells (e.g., glia) when compared to a control AAV (e.g., AAV9) comprising a wild-type AAV9 capsid protein (SEQ ID NO: 9).

In some embodiments, the variant AAV exhibits lower liver cell transduction (e.g., as measured by vector-mediated transgene expression or vector genomes per diploid genomic equivalent) as compared to a control AAV (e.g., AAV9) comprising a wild-type AAV9 capsid protein (SEQ ID NO: 9). In some embodiments, the variant AAV exhibits lower heart cell transduction (e.g., as measured by vector genomes per diploid genomic equivalent) as compared to a control AAV (e.g., AAV9) comprising a wild-type AAV9 capsid protein (SEQ ID NO: 9).

### Pharmaceutical Compositions

In some embodiments, the variant AAV comprising a variant capsid protein comprising an amino acid insertion within loop VIII, wherein the amino acid insertion comprises SEQ ID NO: 15, is provided in a pharmaceutical composition comprising one or more excipients. As used herein, an excipient includes and/or refers to any pharmaceutically acceptable additive, carrier, diluent, adjuvant, or other ingredient, other than the variant AAV vector, which is typically included for formulation and/or administration to a patient. A pharmaceutical composition can comprise a single pharmaceutical formulation or multiple formulations. An excipient further includes and/or refers to an agent that may be added to a formulation to provide a desired consistency (e.g., altering the bulk properties), to improve stability, and/or to adjust osmolality. Examples of commonly used excipients include, but are not limited to, sugars, polyols, amino acids, surfactants, and polymers. In some embodiments, a non-ionic excipient or a non-ionizable excipient, as used herein, includes and/or refers to an agent having no net charge.

In some embodiments, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

### Methods

Provided herein are methods advantageous for delivering a nucleic acid molecule (e.g., a therapeutic nucleic acid) to motor neurons. The advantageous methods utilize variant AAVs comprising a variant capsid protein having an insertion comprising SEQ ID NO: 15 to efficiently and effectively transduce motor neurons.

In some embodiments, provided herein are methods of delivering a nucleic acid molecule to a motor neuron in an individual, the method comprising: administering a variant adeno-associated virus (AAV) comprising the nucleic acid molecule to the individual, wherein the variant AAV comprises a variant capsid protein comprising an amino acid insertion within loop VIII, wherein the amino acid insertion comprises SEQ ID NO: 15.

In some embodiments, provided herein are methods of delivering a therapeutic nucleic acid molecule to a motor neuron in a subject having a motor neuron disease, the method comprising: administering a variant adeno-associated virus (AAV) comprising the therapeutic nucleic acid molecule to the individual, wherein the variant AAV comprises a variant capsid protein comprising an amino acid insertion within loop VIII, wherein the amino acid insertion comprises SEQ ID NO: 15.

In some embodiments, provided herein are methods of treating a motor neuron disease or disorder in an individual, the method comprising: administering a variant adeno-associated virus (AAV) comprising a therapeutic nucleic acid molecule to the individual, wherein the variant AAV comprises a variant capsid protein comprising an amino acid insertion within loop VIII, wherein the amino acid insertion comprises SEQ ID NO: 15. A motor neuron disorder generally refers to and encompasses a condition that disturbs the normal function of motor neurons. In certain embodiments, the motor neuron disorder is characterized by muscle atrophy or wasting and/or fasciculations.

In some embodiments, provided herein are methods of delivering a nucleic acid to a motor neuron, the method comprising: contacting the motor neuron with a variant adeno-associated virus (AAV) comprising the nucleic acid molecule, wherein the variant AAV comprises a variant capsid protein comprising an amino acid insertion within loop VIII, wherein the amino acid insertion comprises SEQ ID NO: 15.

In some embodiments, the motor neurons are upper motor neurons. In some embodiments, the motor neurons are lower motor neurons. In some embodiments, the motor neurons comprise both upper and lower motor neurons. In some embodiments, the motor neurons are in an individual. In some embodiments, the motor neurons are located within the spine. In some embodiments, the motor neurons are located within the ventral horn region of the spinal cord. In some embodiments, the motor neurons are located within the cervical region to the sacral region of the spine. In some embodiments, the motor neurons are located within the cervical region of the spine, the thoracic region of the spine, the lumbar region of the spine, or the sacral region of the spine.

In some embodiments, the variant capsid protein is an AAV serotype 9 (AAV9) capsid protein. In some embodiments, the variant capsid protein without the amino acid insertion comprises 85% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises 95% sequence identity to SEQ ID NO: 9. In some embodiments, the variant capsid protein without the amino acid insertion comprises 90% sequence identity to SEQ ID NO: 9. In some embodiments, the variant capsid protein without the amino acid insertion comprises SEQ ID NO: 9.

In some embodiments, the AAV9 capsid protein comprises an N272A mutation. In some embodiments, the variant capsid protein without the amino acid insertion comprises 85% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises 95% sequence identity to SEQ ID NO: 14. In some embodiments, the variant capsid protein without the amino acid insertion comprises 90% sequence identity to SEQ ID NO: 14. In some embodiments, the variant capsid protein without the amino acid insertion comprises SEQ ID NO: 14.

In some embodiments, the amino acid insertion further comprises a linker. In some embodiments, the amino acid insertion further comprises the formula:

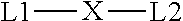

wherein:
L1 comprises a first amino acid linker sequence;
X comprises SEQ ID NO: 15; and
L2 is a second linker sequence.

In some embodiments, L1 and L2 are a different sequence. In some embodiments, L1 and L2 are an identical sequence.

In some embodiments, the variant AAV comprising a variant capsid protein comprising an amino acid insertion within loop VIII, wherein the amino acid insertion comprises SEQ ID NO: 15, exhibits a higher transduction of choline acetyltransferase-positive motor neurons when compared to transduction attainable by an equivalent dose of a control AAV (e.g., AAV9) comprising a wild-type AAV9 capsid protein (SEQ ID NO: 9). In some embodiments, the variant AAV exhibits reduced transduction of choline acetyltransferase-negative neurons and non-neuronal cells (e.g., glia) when compared to a control AAV (e.g., AAV9) comprising a wild-type AAV9 capsid protein (SEQ ID NO: 9).

In some embodiments, the variant AAV exhibits increased transduction of upper motor neurons when compared to transduction attainable by an equivalent dose of a control AAV (e.g., AAV9) comprising a wild-type AAV9 capsid protein (SEQ ID NO: 9). In some embodiments, the variant AAV exhibits reduced transduction of non-neuronal cells (e.g., glia), when compared to a control AAV (e.g., AAV9) comprising a wild-type AAV9 capsid protein (SEQ ID NO: 9).

In some embodiments, the variant AAV transduces a higher ratio of choline acetyltransferase-positive motor neurons to choline acetyltransferase-negative neurons and non-neuronal cells (e.g., glia) when compared to a control AAV (e.g., AAV9) comprising a wild-type AAV9 capsid protein (SEQ ID NO: 9). In some embodiments, the variant AAV transduces a higher ratio of upper motor neurons to other types of neurons and non-neuronal cells (e.g., glia) when compared to a control AAV (e.g., AAV9) comprising a wild-type AAV9 capsid protein (SEQ ID NO: 9).

In some embodiments, the variant AAV exhibits lower liver cell transduction (e.g., as measured by vector genomes per diploid genomic equivalent) as compared to a control AAV (e.g., AAV9) comprising a wild-type AAV9 capsid protein (SEQ ID NO: 9). In some embodiments, the variant AAV exhibits lower heart cell transduction (e.g., as measured by vector genomes per diploid genomic equivalent) as compared to a control AAV (e.g., AAV9) comprising a wild-type AAV9 capsid protein (SEQ ID NO: 9).

In some embodiments, the variant AAV is administered by injection into the individual. In certain embodiments, the injection is intravenous injection.

As used herein, individual is synonymous with patient and/or subject and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

As used herein, treating or treatment of includes and/or refers to ameliorating the disease or disorder or symptoms thereof (e.g., slowing or arresting or reducing the development of the disease or at least one of the clinical symptoms thereof). In certain embodiments, treating or treatment also includes and/or refers to alleviating or ameliorating at least one physical and/or biological parameters including those which may not be discernible by the patient. In certain embodiments, treating or treatment includes and/or refers to modulating a disease, disorder, or biological process either physically, (e.g., stabilization of a discernible symptom), physiologically, (e.g., stabilization of a physical and/or biological parameter), or both. In certain embodiments, treating or treatment includes and/or refers to preventing or delaying the onset or development or progression of the disease or disorder. In certain embodiments, treating or treatment includes and/or refers to preventing or delaying or inhibiting the deterioration of (i) a healthy physiological state or (ii) a baseline physiological state (e.g., the progression of a disease or disorder).

As used herein, a sample includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As used herein, in any instance or embodiment described herein, “comprising” may be replaced with “consisting essentially of” and/or “consisting of”. used herein, in any instance or embodiment described herein, “comprises” may be replaced with “consists essentially of” and/or “consists of”.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

### EXAMPLES

### Example 1—Motor Neuron Targeting

AAV-CAP2, an adeno-associated virus (AAV) comprising the nucleic acid molecule to the individual, wherein the variant AAV comprises a variant capsid protein comprising an amino acid insertion within loop VIII, wherein the amino acid insertion comprises SEQ ID NO: 15 (e.g., SEQ ID NO: 16), is a novel AAV capsid identified by the TRAnscription-dependent Directed Evolution (TRADE) platform, exhibits enhanced motor neuron tropism following vector administration in both rodents and non-human primates (NHPs).

Cynomolgus macaques and C57BL/6 mice were treated with a single intravenous injection of an AAV-CAP2-CAG-GFP vector (an AAV vector comprising a nucleic acid molecule encoding GFP and having a variant capsid protein comprising an amino acid insertion within loop VIII). Data was collected from animals 3 weeks after injection and tissues were collected for downstream histological and biodistribution analyses. To evaluate the efficiency and specificity of motor neuron transduction with the CAP2 variant, the phenotype of the GFP-positive transduced cells with triple immunolabeling utilizing selective pan-neuronal (NeuN, neuronal nuclei) and motor neuron markers (SMI-32 and choline acetyltransferase, ChAT) was characterized. Based on previous histological descriptions, Betz cells in the NHP primary motor cortex were identified by a set of criteria: their soma size (>600 μm), localization (5b sublayer), proximal dendritic morphology and SMI-32 immunoreactivity.

AAV-CAP2 demonstrated a significant bias for transducing SMI-32-immunopositive upper motor neurons with minimal glia transduction in both species. In the NHP ventral horn, the ChAT immunostaining revealed that a large number of motor neurons expressed GFP homogeneously all along the ventral horn from the cervical to the lumbar segments. The GFP signal was also present in the axons of the sciatic nerve indicating a transport of the GFP protein along the peripheral axonal pathway. The GFP-positive motor neurons were scored in the cervical and lumbar enlargements that innervate upper and lower limbs, respectively. Motor neuron quantification revealed that >80% of the lower motor neurons expressed GFP in the cervical and lumbar spinal cord segments of the vector-injected NHPs. Notably, no obvious GFP signal was detected in ChAT-negative cells, supporting the motor neuron tropism of the CAP2 variant. Spinal motor neuron transduction was also evident in mice but to a lesser extent compared to NHPs, suggesting possible cross-species differences. The data demonstrates that AAV-CAP2 is a powerful vector for the use in the treatment of a wide number of motor neuron diseases.

### Aim

The initial challenge was to develop a novel AAV capsid that could transduce neurons in the brain and spinal cord via intravenous (i.v.) delivery. AAV9 was selected as the backbone. The AAV9 backbone was modified with an N272A mutation, which had previously been determined to confer a liver detargeting phenotype (data not shown). A pAAV9-N272A-hSYN1-TRADE plasmid was constructed containing two expression cassettes oriented antisense to each other and flanked by the AAV2 inverted terminal repeats (ITRs). On one side of the genome, the AAV2 p40 promoter and the AAV2 viral genome intron are followed by the AAV9-N272A cap gene and the AAV2 polyadenylation signal. On the other side of the genome and antisense to the p40-driven cap ORF, an overlapping transgene cassette was used that was consisted of human synapsin I (hSYN1) promoter-enhancer sequence, a truncated minute virus of mice (MVM) intron, the non-coding antisense cap sequence, and an SV40 polyadenylation signal placed in the AAV2 intron (FIG. 1). The hSYN1 promoter-enhancer element was selected to drive expression of anti-sense mRNA in neurons in vivo, which can then be used to identify capsid mutants that exhibit enhanced neuronal tropism. The initial TRADE peptide display plasmid library (pAAV9-N272A-hSYN1-TRADE-Lib0) was constructed by PCR amplification of an AAV9 cap sequence harboring a loop VIII [NNK]8 (GGGS[NNK]8GGGGS) substitution at position Q588 of the AAV9N272A VP1. The plasmid library was used to create a corresponding virus library using the standard triple transfection production method. FIG. 1 shows a diagram of a TRADE construct.

### In Vivo Selection

Initial in vivo selection was carried out in 8-week-old C57BL/6J male mice. The AAV9-N272A-hSYN1-TRADE-Lib0 vector was administered into two mice and brain tissue was harvested 12 days post-injection. In this first round selection, a brain region that was relatively enriched for neurons was used and thus extracted RNA from crudely dissected frontal cortex. Total RNA was isolated from each animal independently, mixed in a 1:1 ratio and identified recovered sequences by RT-PCR. The region containing the peptide insertion was assembled in a TRADE vector backbone to generate a plasmid library representing capsid sequences that had undergone one round of selection (i.e., pAAV9-N272A-hSYN1-TRADE-Lib1). This plasmid library was used to generate the corresponding AAV library and the process was repeated for a total of 3 rounds of selection in mice. Rounds 2 and 3 were carried out in a similar manner to the first round with a few modifications. In the latter rounds, doses ranging from 1×10⁹ to 1×10¹² vg per mouse in 10-fold dilutions (n=2 animals per dose) were administered, but only samples from the animals administered 1×10¹¹ vg dose were carried forward. In parallel, a single round of selection was performed using a male rhesus macaque. The AAV9-N272A-hSYN1-TRADE-Lib0 vector was administered intravenously and the animal was euthanized 14 days post-injection. Tissues were collected and frozen on dry ice. RT-PCR amplicons of the antisense cap mRNA containing the peptide fragment were recovered from RNA extracted from the brain and sub-cloned into a plasmid backbone and made into a plasmid library. DH10B cells were then transformed with the plasmid library, and individual clones were miniprepped and sent for Sanger sequencing. The top five most common variants following three rounds of selection in C57BL/6J mice were selected, as well as 21 variants that were recovered by Illumina sequencing from various regions of the rhesus macaque brain after a single round of selection, for AAV DNA/RNA Barcode-Seq analysis.

### AAV DNA/RNA Barcode-Seq Analysis

Viral barcode clones were individually packaged into the novel, TRADE-identified, capsids using the standard triple transfection method. In addition, barcoded vectors in key reference capsids including AAV9, AAV9-N272A (parental) and AAV-PHP.B were generated. Crude lysate titers of DNase-resistant vector genomes were determined by a quantitative dot blot assay using a probe against the hSYN1 promoter. Each barcoded viral clone was then mixed at an approximately equimolar ratio based on the dot blot titers and the pooled crude lysate was purified as a library to generate the scAAV-hSYN1-GFP-BCLib.

The scAAV-hSYN1-GFP-BCLib library was administered into a single male rhesus macaque at a dose of 2×10¹³ vg/kg and C57BL/6J mice (n=3) at a dose of 1.7×10¹³ vg/kg and harvested tissues two weeks post-injection. AAV RNA Barcode-Seq using tissue dissected from a variety of brain regions was performed. From this analysis, AAV-CAP2 was selected for additional characterization (data not shown).

### AAV-CAP2 Single Capsid Validation in NHPs

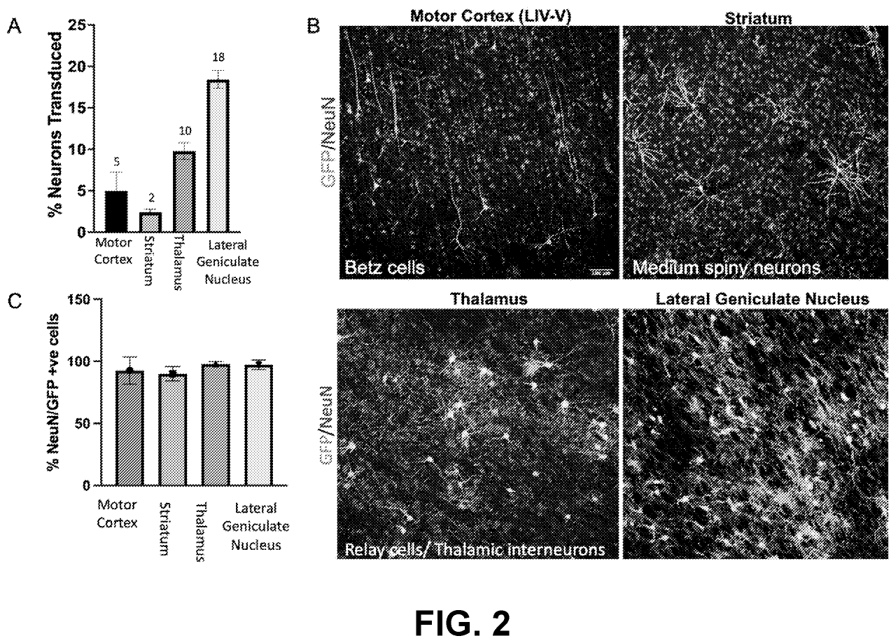

To assess the ability of AAV-CAP2 to transduce neural cells an enhanced green fluorescent protein (GFP) under the regulation of the ubiquitous CAG promoter-enabling characterization across cells throughout the body—was packaged in AAV-CAP2. The GFP reporter employed bore an N-terminal nuclear localization signal (nls) that allows nuclear enrichment to some degree while retaining cytoplasmic signals. Two cynomolgus macaques were treated with a single i.v. injection of the AAV-CAP2-CAG-GFP vector at a dose of 5×10¹³ vg/kg. Animals were euthanized 3 weeks after injection and tissues were harvested for downstream biodistribution and histological analyses. Immunostaining with an anti-GFP antibody revealed robust and broad GFP expression across multiple brain regions and neuronal subtypes in the adult NHP brain with strongest expression observed in the motor cortex and thalamic regions (FIG. 2A-B). To determine neuronal transduction efficiency and specificity of the transduced cells, double-immunofluorescence microscopy analysis was performed in which GFP expression was coupled with a pan-neuronal marker, NeuN (Neuronal Nuclei) (FIG. 2B). This analysis revealed that neurons accounted for 93% of the transduced cells in the motor cortex, 90% in the striatum and 97% in the thalamus and lateral geniculate nucleus (FIG. 2C). Glial transduction measured by co-staining with an astrocytic marker, GFAP, and an oligodendrocyte marker, Olig2, was minimal (data not shown).

Upper Motor Neuron Transduction with AAV-CAP2

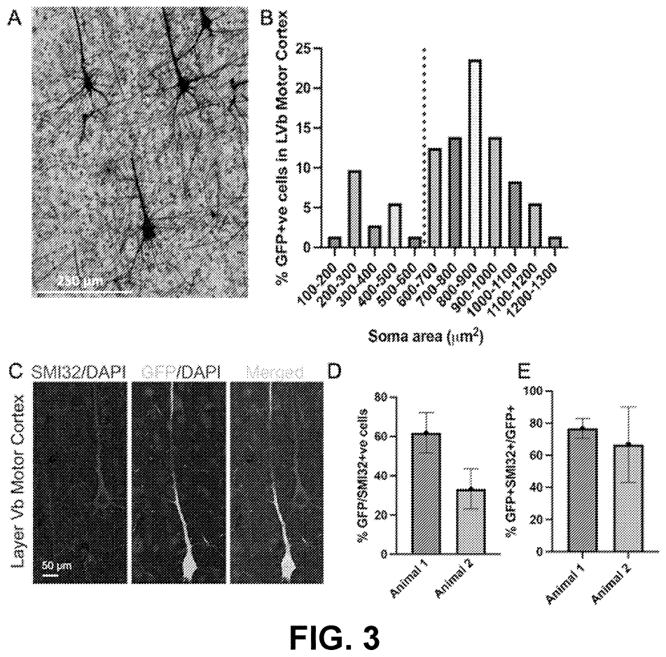

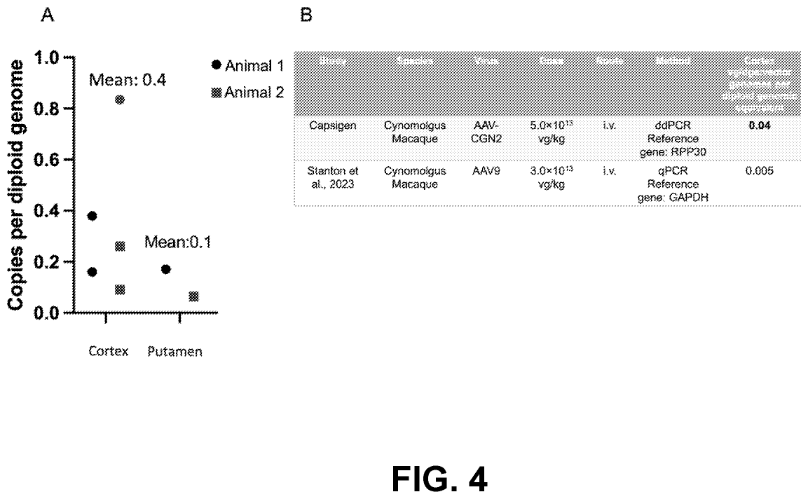

Interestingly, in the frontal cortex, the majority of the transduced cells were located throughout the layer Vb of the primary motor cortex and exhibited an upper motor neuron morphology (FIG. 3A). Upper motor neurons (UMN) refer to Betz gigantopyramidal cells in NHPs and humans or corticospinal neurons in mice synapsing with the lower motor neurons (LMNs) in the ventral horns of the spinal cord (Menon and Vucic 2021; Braak and Braak 1976; Rivara et al. 2003). Based on previous histological descriptions, Betz cells in the NHP primary motor cortex can be identified by a set of criteria: their soma size (>600 μm), localization (Vb sublayer), proximal dendritic morphology and SMI-32 immunoreactivity (Braak and Braak 1976; Jacobs et al. 2018; Meyer 1987; Rivara et al. 2003; Szocsics et al. 2021). To evaluate the efficiency and specificity of upper motor neuron/Betz cells transduction, a morphometric analysis was performed of the GFP-positive cells in the layer Vb of the primary motor cortex (FIG. 3B). The distribution of the soma size of the transduced cells exhibited a bimodal patten, with one peak at 200-300 μm² and another peak at 800-900 μm² (FIG. 3B). Making a categorical distinction of soma size between Betz and pyramidal cells at 600 μm², the majority of the transduced cells in Layer Vb in the motor cortex had a soma size within the 600-1300 μm² range (FIG. 3B). The phenotype of these large GFP-positive transduced cells was next characterized in the motor cortex with double immunofluorescence microscopy against GFP and a selective upper motor neuron marker, SMI-32 (FIG. 3C). Strikingly, AAV-CAP2 transduced 48% of all giant SMI-32-positive Betz cells in NHP motor cortex and 71% of all transduced cells in Layer Vb were SMI-32-positive Betz cells. Vector copy numbers in the motor cortex and putamen were determined for both animals using ddPCR (FIG. 4A). The GFP quantitation data obtained by ddPCR was assured by quantifying a genomic reference in the Ribonuclease P protein subunit p30 (RPP30) locus, to determine the number of vector genome copy numbers per diploid genome. The average vector genome copy numbers per diploid genome in the cortex and putamen were of 0.4 and 0.2 respectively (FIG. 4A). Although motor neuron transduction or biodistribution data is not available in the literature, a recent publication (Stanton et al. 2023) reported a vector copy number of 0.05 in motor cortex following AAV9 i.v. administration at a dose of 3×10¹³ vg/kg (FIG. 4B). If a 3×10¹³ vg/kg dose was used in the study, the estimated copy number would correspond to 0.009 vg/diploid genome, which is ~4 fold lower than observed assuming a linear dose response. Given that AAV9 transduces more glial cells than neurons in the NHP brain, the estimated copy number within the NHP brain neurons in Stanton et al.'s study should be less than 0.009 vg/diploid genome, highlighting the superiority of AAV-CAP2 over AAV9 in neuronal transduction in NHPs.

Lower Motor Neuron Transduction with AAV-CAP2

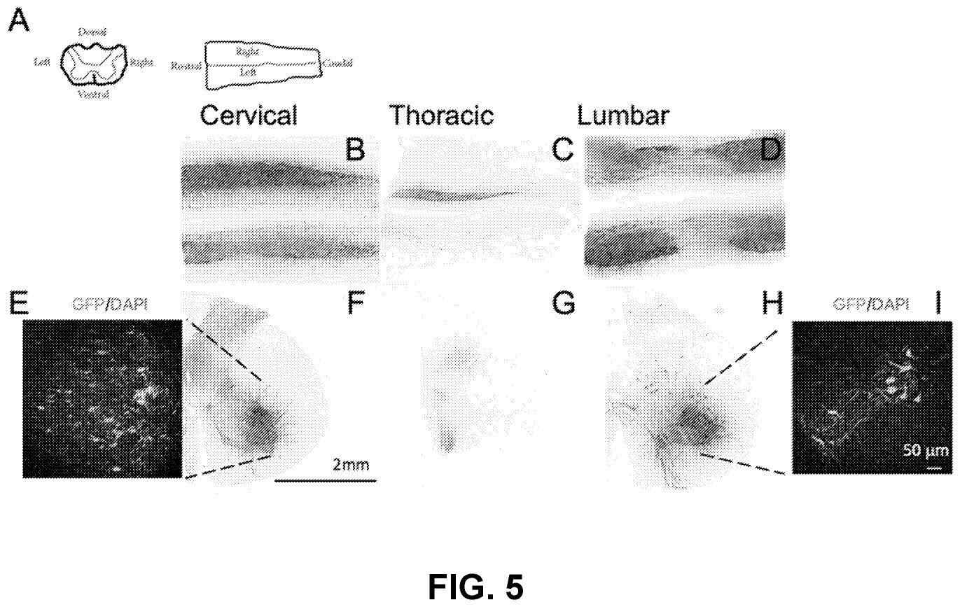

Given the increased potency of AAV-CAP2 in upper motor neuron transduction, the capabilities of AAV-CAP2 to demonstrate enhanced transduction in the spinal cord was examined. A single i.v. injection of AAV-CAP2-CAG-GFP resulted in transduction of neurons along the entire length of the spinal cord. Robust GFP signal was detected in cells with motor neuron morphology and their axons at the cervical (FIGS. 5B, F and E), thoracic (FIGS. 5C and G) and lumbar (FIGS. 5D, H and I) levels.

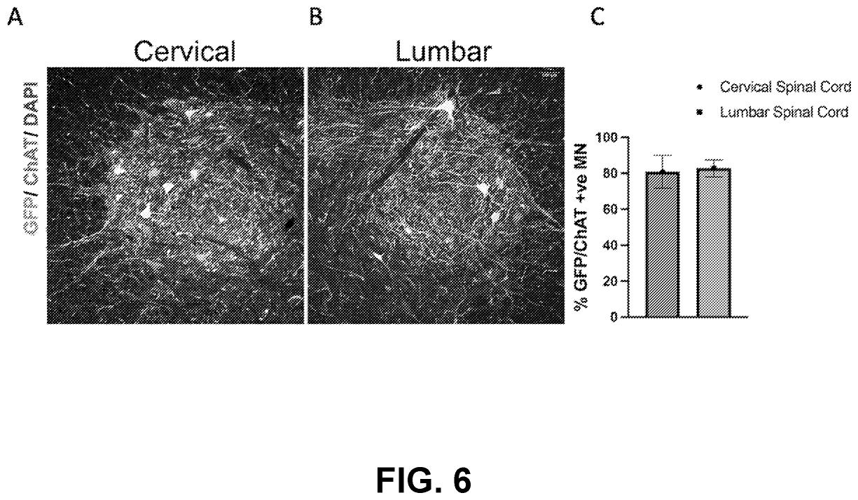

To identify these GFP-positive cells as lower motor neurons, immunostaining was performed for ChAT (choline acetyltransferase), an enzyme that catalyzes acetylcholine synthesis and serves as a selective marker for cholinergic motor neurons in the ventral horn (Stifani 2014). ChAT immunostaining demonstrated that the majority of the motor neurons expressed GFP in both the cervical (81%) and the lumbar (83%) segments, innervating upper and lower limbs, respectively (FIG. 6, A-C) (Stifani 2014).

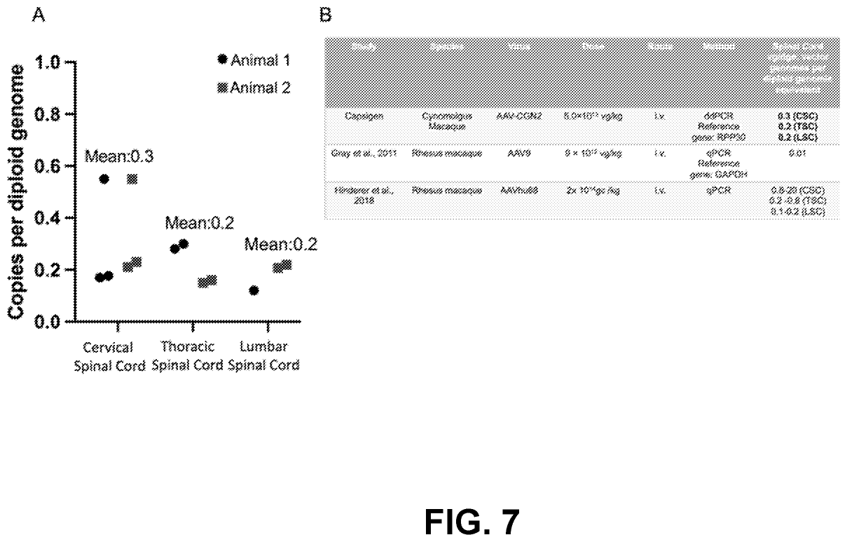

GFP transgene-specific quantitative PCR performed in the cervical, thoracic and lumbar spinal cord segments of the two injected macaques showed equal or higher numbers of vg per diploid genome in the spinal cord compared to the reported numbers in the literature (FIG. 7) (Gray et al. 2011; Hinderer et al. 2018).

Dorsal Root Ganglia Transduction with AAV-CAP2

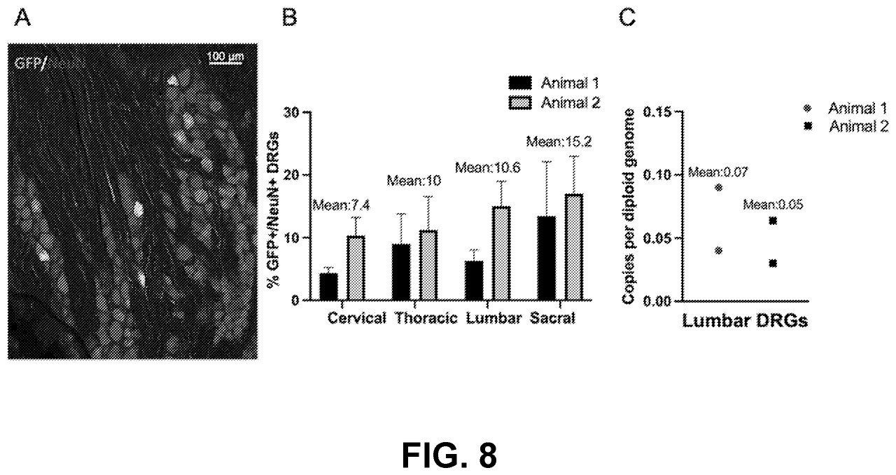

High transduction of the DRGs following AAV vector administration has been associated with neuroinflammation that can result in serious neurotoxicity. DRG transduction was assessed 21 days after i.v. administration of AAV-CAP2-CAG-GFP (FIG. 8). Immunostaining with antibodies against GFP (green) and a neuronal marker, NeuN (red), showed 7%-15% mean transduction in DRGs throughout the spine (FIGS. 8A and B). Vector genome copy numbers per diploid genome was determined in the DRGs, showing 0.05-0.03 (FIG. 8C).

The spinal cord and DRG of the two NHPs was assessed for abnormal pathology, including mononuclear cell infiltration and neuronal cell body degeneration in the DRG and secondary axonopathy in the spinal cord. The immune responses in multiple DRG sections from the cervical, thoracic, and lumbar regions of the spine from both animals were analyzed on hematoxylin and eosin (H&E)-stained histology sections by an experienced histopathologist. The severity scores for each individual DRG segment were established using a scale developed by Hordeaux (Hordeaux et al. 2020). Only mild lymphocyte infiltration was reported in the DRGs and no secondary axonopathy was detected in the spinal cord of both animals (data not shown).

### Biodistribution in Peripheral Organs

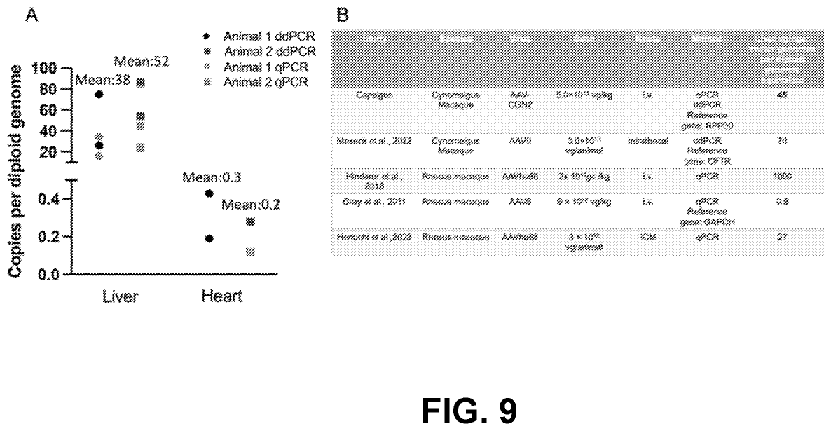

From the same animals, portions of the liver and heart were used for biodistribution analysis and quantitation of vector genomes by quantitative PCR (qPCR and ddPCR) (FIG. 9A). A comparison of data of the vector genome copy numbers in the liver with the data reported in the literature (FIG. 9B) indicated that, with the exception of the study done by Gray et al. (Gray et al. 2011) who reported a surprisingly low liver vector genome copy number, AAV-CAP2 demonstrated lower vector genome abundance in the liver relative to that of AAV9 (Gray et al. 2011; Hinderer et al. 2018; Horiuchi et al. 2022; Meseck et al. 2022).

### Single Capsid Validation of AAV-CAP2 in Mice

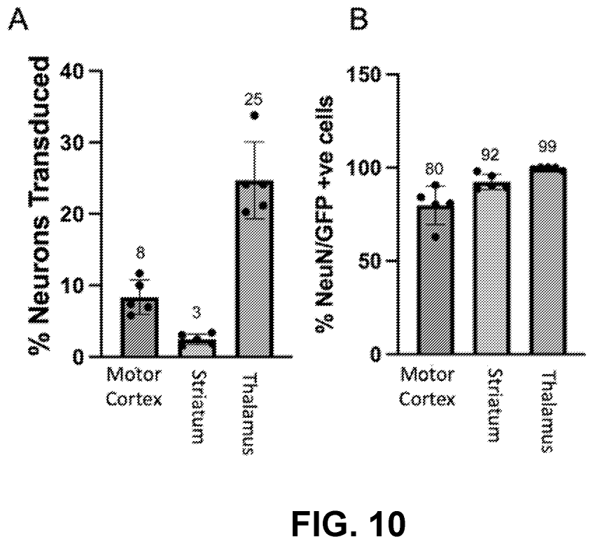

Our findings demonstrate that widespread and strong upper and lower motor neuron transduction is achievable by single i.v. injection of AAV-CAP2 in NHPs. To determine whether systemic delivery of AAV-CAP2 in mice would also result in enhanced neuronal transduction as observed in NHPs, 5×10¹³ vg/kg of AAV-CAP2-CAG-GFP was injected, via the tail vein, in 8-week-old C57BL/6 male mice. Three weeks after injection, tissues were harvested and assessed for neuronal transduction and vector biodistribution. The results showed that AAV-CAP2 vector transduced a high fraction of neurons (measured by co-staining with NeuN) in all tested brain regions across the mouse central nervous system (CNS). Neuronal transduction rates varied between regions; 8% of neurons in the motor cortex colocalized with GFP signal, 25% in the thalamus while this rate dropped to 3% in the striatum (putamen) (FIG. 10A). AAV-CAP2's attribute of high neuronal specificity was found to also be retained in mice, showing 80-99% neuronal specificity across different regions (FIG. 10B).

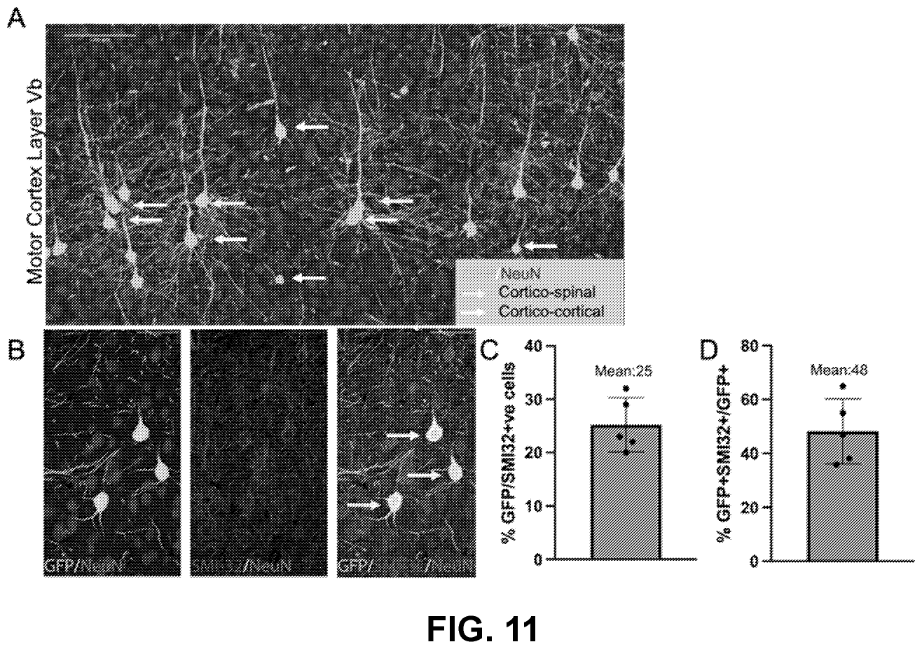

Whether the motor neuron tropism of the AAV-CAP2 vector found in the NHPs was also retained in mice was examined. An equivalent upper and lower motor neuron transduction analysis was performed in mice. First, AAV-CAP2-CAG-GFP transduced cells with neuronal morphology along the “motor strip” in Layer Vb of the murine primary motor cortex (FIG. 11A) was observed. This profile was similar to findings in the NHPs. Further upper motor neuron transduction efficiency analysis confirmed that the target specificity of the AAV-CAP2 vector was biased towards SMI-32-immunopositive upper motor neurons also in mice (FIG. 11B). AAV-CAP2 targeted 25% of the SMI-32-positive upper motor neurons, while 45% of all the transduced cells in the murine primary motor cortex were upper motor neurons (FIG. 11B-D).

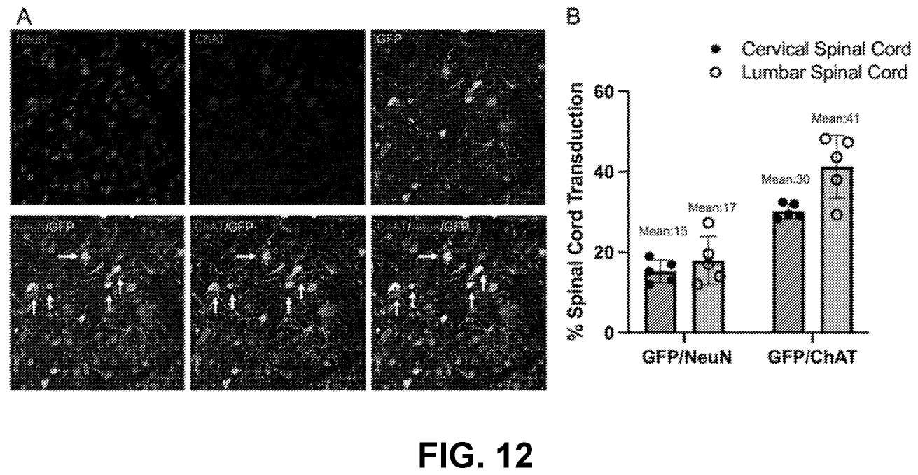

Next, target specificity of the AAV-CAP2 in the murine spinal cord (FIG. 12) was examined. Triple staining with GFP, ChAT and NeuN revealed colocalization of ChAT-immunolabeled motor neurons (MN) with GFP and NeuN in the ventral horn (FIG. 12A). Scoring GFP-positive motor neurons in the cervical and lumbar segments showed that 30% of cervical spinal motor neurons and 41% of lumbar spinal motor neurons were transduced in mice. Enhanced spinal motor neuron transduction was observed albeit lower in mice compared to NHPs (~80%). This observation may indicate that there is a cross-species difference in transduction to some extent.

### Comparison of AAV-CAP2 Transduction in Mice and NHPs

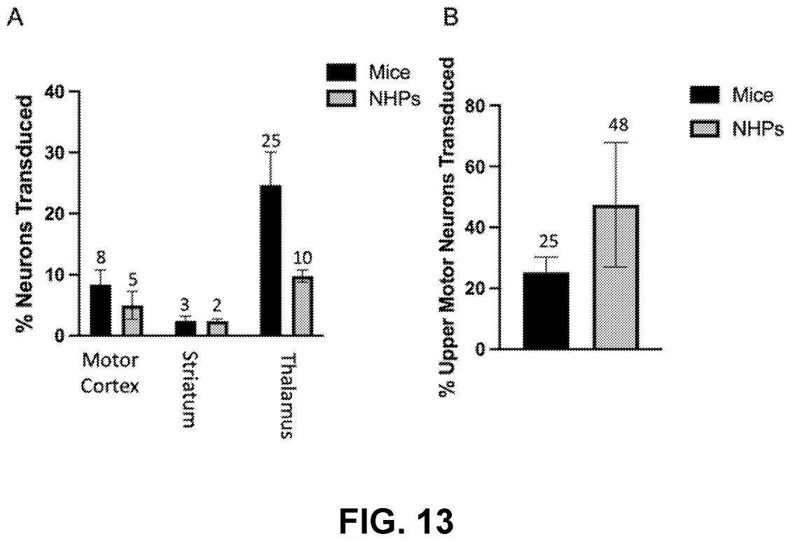

Target specificity of the AAV-CAG-CAP2-GFP vector was analyzed for the brain of mice and cynomolgus macaques. Comparable levels of transduced neurons and in the brains of mice and NHPs (FIG. 13A) was observed. Additionally, the efficiency of upper motor neuron transduction did not differ between the two species (FIG. 13B) further supporting the motor neuron tropism of this capsid.

### Discussion

Upper and lower motor neurons are the distinct neuronal subtypes showing primary neurodegeneration in motor neuron diseases. Therefore, efficient transduction of upper and lower motor neurons is critical for any successful gene therapy approach. Systemic administration of viral vectors could serve as an optimal route of administration; however inefficient crossing of the blood-brain barrier, poor target cell specificity, and high uptake by the liver remain challenges with commonly used AAV serotypes. In particular, targeting upper motor neurons is challenging given their relatively low abundance within the brain and specific location within layer Vb of the cortex. Even though retrograde labeling studies in mice showed that it is indeed possible to use AAV to retrogradely transduce motor neurons and induce selective gene expression, these approaches have significant limitations as they only target the motor neurons that innervate specific muscle groups (Genc et al. 2022; Jara et al. 2014). Additionally, there are currently no approaches for selective Betz cell transduction in the motor cortex of NHPs and humans. In this respect, AAV-CAP2 shows a significant bias for transducing upper motor neurons with minimal glia transduction in both mice and NHPs.

Efficient systemic spinal cord transgene delivery to lower motor neurons is also central to the success of gene therapy and the genetic manipulation for motor neuron diseases. Previous studies (Hinderer et al. 2018) reported that 30-90% of motor neuron transduction at each level of the spinal cord in rhesus macaques could be achieved following systemic administration of an AAV9 variant, AAVhu68, at a dose of 2×10¹⁴ vg/kg, which is 4-fold higher than the dose used in this study. However, unexpected toxicities including acute systemic inflammation, coagulation defects and hepatic toxicity were encountered following such a high dose (Hinderer et al. 2018). Intrathecal delivery has been shown to be less affected by preexisting antibodies, which are lower in cerebral spinal fluid. Thus, the intrathecal approach has been employed for transduction in the brain and spinal cord of NHPs, although transduction efficiency still needs to be improved to reach therapeutic levels (Gray et al. 2013). Lumbar intrathecal injection of AAV-F, another AAV9 variant, in cynomolgus macaques resulted in a gradient of lower motor neuron transduction from lumbar (higher) to cervical (lower), ranging from 70% to 40% respectively (Beharry et al. 2022). In mice, i.v. delivery of AAV9 in adult animals achieved an average lower motor neuron transduction rate of ~19% (Duque et al. 2009). The observation that intravenous delivery of 5×10¹³ vg/kg AAV-CAP2 vector in adult animals resulted in transduction of ~35% of spinal motor neurons in mice and ~80% of spinal cord motor neurons in NHPs with low toxicity, makes AAV-CAP2 an attractive capsid for use in gene therapies to treat motor neuron diseases. The molecular mechanisms of the motor neuron tropism of the AAV-CAP2 vector are currently unknown but they could result from a combination of capsid amino acid differences and specific receptor or coreceptor interactions distinct from that of other AAV capsids, which warrants further investigation.

In summary, the above-described study has identified AAV-CAP2, a novel AAV capsid that transduces both upper and lower motor neurons with high efficiency and specificity in both mice and NHPs, using TRAnscription-dependent Directed Evolution (TRADE). The data suggests that AAV-CAP2 offers a best-in-class capsid for the treatment of a wide range of motor neuron disorders.

### Methods

Non-human primates: Non-human primate studies were performed at Envol Biomedical (Florida, USA) in accordance with their standard operating protocols and procedures approved by their IACUC. Two healthy and treatment-naïve (one male and one female) cynomolgus macaques, approximately two years of age housed in standard conditions at Envol Biomedical, were selected for the AAV-CAP2 study. Both animals were verified to have a serum AAV2 and AAV9 neutralizing antibody titer of less than 1:5. Macaques received an i.v. bolus injection of AAV-CAP2-CAG-GFP at a dose of 5×10¹³ vg/kg. Animals were euthanized and perfused with PBS, after which CNS and other organ tissues were harvested. Brains were removed and hemisected with one hemisphere immersion-fixed overnight in 4% paraformaldehyde and the other hemisphere dissected to separate brain regions. CNS regions, as well as peripheral organs, were snap frozen and stored at −80° C. for molecular processing.

NHP neurohistology embedding, sectioning and staining: Two NHP brain hemispheres and all segments of spinal cords and DRGs were processed for embedding and sectioning at NeuroScience Associates, (NeuroScience Associates, Knoxville, TN). The samples were treated overnight with 20% glycerol and 2% dimethyl sulfoxide in phosphate-buffered saline (PBS) to prevent freeze artifacts. The specimens were then embedded, with 2 brain hemispheres from the 2 animals for coronal embedding, 16 cords segments for transverse and coronal embedding and 8 DRG segments were also embedded. The samples were arranged in a gelatin matrix for each block using MultiBrain®/MultiCord® Technology. The MultiBrain®/MultiCord® blocks were sectioned with a setting on the microtome of 40 μm.

DAB-GFP immunohistochemistry: For immunohistochemistry (IHC), brain sections at every twenty-fourth section (at an interval of 960 microns), spinal cord sections at every twentieth section (at an interval of 800 microns) and DRGs sections at every tenth section (at an interval of 400 microns), was stained using a free-floating method. All incubation solutions from the primary antibody onward used Tris-buffered saline (TBS) with Triton X100 as the vehicle; all rinses were with TBS. After a hydrogen peroxide treatment and rinses, the sections were immunostained with the primary antibodies overnight at room temperature, as shown in the table below. Vehicle solutions contained Triton X100 for permeabilization. Following rinses, a biotinylated secondary antibody (anti-IgG of the host animal in which the primary antibody was produced) was applied. After further rinses, Vector Lab's ABC solution Catalog #PK-6100 (avidin-biotin-HRP complex; a dilution of 1:222 was applied). The sections were again rinsed, then treated with a chromogen, diaminobenzidine tetrahydrochloride (DAB), nickel (II) sulfate and hydrogen peroxide to create a visible reaction product. Following further rinses, the sections were mounted on gelatin-coated glass slides, then air dried. The mounted slides were counterstained with a full Thionine Nissl staining.

Full Thionine Nissl counterstaining: the air dried mounted stained slides were carried through the following sequence: 95% ethanol, 95% ethanol/formaldehyde; 95% ethanol, 70% ethanol, deionized water (dH₂O). The slides were then stained in a Thionine solution made in an acetate buffer, pH4.5, rinsed in dH₂O and visually assessed. Following the dH₂O rinses, the slides were dehydrated in alcohols, cleared in xylene and coverslipped with Permount (Fisher Scientific, Pittsburgh PA).

Mice: Eight-week-old healthy and treatment-naive male C57BL/6J (JAX, #000664) mice were purchased from the Jackson Laboratory. Mice were housed in standard conditions in accordance with the IACUC at Oregon Health and Science University. In all mouse, AAV vector administration was performed via the tail vein injection. Tissue samples were collected from the mice after whole body perfusion with either phosphate-buffered saline (PBS) only or PBS followed by 4% paraformaldehyde (PFA) depending on the downstream experiments.

Mice neurohistology, embedding and sectioning: Brain and spinal cord, together with the DRGs were sampled and post-fixed in 4% PFA overnight at 4° C. Brains were sectioned in the coronal place at a thickness of 40 μm using a Vibratome (Leica VT1000, Wetzlar, Germany) and kept as free-floating sections. Spinal cord and DRG sections were cut in the coronal plane and transverse plane at 40-μm thickness on a cryostat (Microm HM550, Thermo Fisher Scientific, Waltham, MA) and mounted on amino silane-coated slides.

Immunofluorescence staining: Sections were washed three times with TBS. Subsequently, they were incubated with 0.3% Triton X-100 in TBS containing 5% normal goat serum (NGS) for 1 h at room temperature, followed by incubation with a primary antibody, diluted in 2% NGS and 0.3% Triton X-100 in TBS overnight at 4° C. Then, the sections were washed with PBS three times (3×10 min) and incubated with a secondary antibody, diluted in in PBS for 1 h at room temperature. They were then washed in PBS (3×10 min) and mounted with DAPI Fluoromount-G (SouthernBiotech). Control sections were stained using the same protocol but omitting the primary antibodies. All processes were performed in a dark chamber.

The primary antibodies were as follows: Chicken anti-GFP (AVES, AB_2307313, 1:500), rabbit anti-NeuN (Millipore, ABN78, 1:500), goat anti-choline acetyltransferase (ChAT; Millipore, AB144P, 1:100), mouse anti-SMI-32 (Biolegend, 801702, 1:100), rabbit anti-GFAP (DAKO, Z0334, 1:1000). The secondary antibodies used were as follows: goat anti-chicken immunoglobulin G (IgG; Abcam, Alexa488), goat anti-rabbit IgG (Molecular Proves, Alexa568), goat anti-rabbit IgG (Molecular Proves, Alexa647), goat anti-mouse IgG (Molecular Probes, Alexa555, Eugene, OR, USA), donkey anti-goat IgG (Molecular Probes, Alexa647) at 1:300.

Imaging and quantification of transduction: For imaging, an LSM 9 MP laser scanning microscope (Carl Zeiss MicroImaging, Thornwood, NY) was used. All quantification analyses were performed in a blinded fashion. Viral tropism analysis was conducted using Cell Counter probe in ImageJ and Imaris software. Images from immunofluorescently labeled, level-matched sections (4 to 8 per animal) across motor cortex, putamen, thalamus, spinal cords and DRGs segments were acquired using a 20× lens at equivalent acquisition settings. Cellular tropism is reported as a percentage of total GFP-positive cells counted (minimum 50 cells per each of the replicates). Statistical analyses were based on the average numbers for each animal. All statistical analyses were performed using Prism software (GraphPad Software Inc., La Jolla, CA, USA)

Quantification of vector genome copy numbers: Genomic DNA was extracted and purified from different tissues (i.e., brain, spinal cord, DRGs, liver and heart) using the QIAamp DNA tissue kit (Qiagen, Hilden, Germany). The extracted DNA was analyzed for yield and purity using a NanoDrop One UV/Vis spectrophotometer (Thermo Scientific, Wilmington, MA, USA).

To measure the viral genome copy numbers, one ug of gDNA was digested with Bam HI, which does not cut the viral genome. The digested gDNA samples were diluted to 100 ng, 50 ng, 25 ng and 12.5 ng. Twenty μL ddPCR reaction mixtures were prepared for each dilution using the 2× digital PCR supermix for probes (No dUTP) and primer/probe targeting the GFP region (VIC) and the RPP30 gene (FAM). The GFP quantitation data obtained by ddPCR was assured by quantifying a genomic reference in the Ribonuclease P protein subunit p30 (RPP30) locus, to determine the number of vector genome copy numbers per diploid genome. An average of all four dilutions were plotted.

SEQUENCES

| SEQ ID NO: | SEQUENCE | Name |
|---|---|---|
| 1 | MAADGYLPDWLEDNLSEGIREWWDLKPGAPKPKANQQKQDDGRGLV | AAV1 |
| | LPGYKYLGPFNGLDKGEPVNAADAAALEHDKAYDQQLKAGDNPYLR | |
| | YNHADAEFQERLQEDTSFGGNLGRAVFQAKKRVLEPLGLVEEGAKT | |
| | APGKKRPVEQSPQEPDSSSGIGKTGQQPAKKRLNFGQTGDSESVPD | |
| | PQPLGEPPATPAAVGPTTMASGGGAPMADNNEGADGVGNASGNWHC | |
| | DSTWLGDRVITTSTRTWALPTYNNHLYKQISSASTGASNDNHYFGY | |
| | STPWGYFDFNRFHCHFSPRDWQRLINNNWGFRPKRLNFKLFNIQVK | |
| | EVTTNDGVTTIANNLTSTVQVFSDSEYQLPYVLGSAHQGCLPPFPA | |
| | DVFMIPQYGYLTLNNGSQAVGRSSFYCLEYFPSQMLRTGNNFTFSY | |
| | TFEEVPFHSSYAHSQSLDRLMNPLIDQYLYYLNRTQNQSGSAQNKD | |
| | LLFSRGSPAGMSVQPKNWLPGPCYRQQRVSKTKTDNNNSNFTWTGA | |
| | SKYNLNGRESIINPGTAMASHKDDEDKFFPMSGVMIFGKESAGASN | |
| | TALDNVMITDEEEIKATNPVATERFGTVAVNFQSSSTDPATGDVHA | |
| | MGALPGMVWQDRDVYLQGPIWAKIPHTDGHFHPSPLMGGFGLKNPP | |
| | PQILIKNTPVPANPPAEFSATKFASFITQYSTGQVSVEIEWELQKE | |
| | NSKRWNPEVQYTSNYAKSANVDFTVDNNGLYTEPRPIGTRYLTRPL | |
| 2 | MAADGYLPDWLEDTLSEGIRQWWKLKPGPPPPKPAERHKDDSRGLV | AAV2 |
| | LPGYKYLGPFNGLDKGEPVNEADAAALEHDKAYDRQLDSGDNPYLK | |
| | YNHADAEFQERLKEDTSFGGNLGRAVFQAKKRVLEPLGLVEEPVKT | |
| | APGKKRPVEHSPVEPDSSSGTGKAGQQPARKRLNFGQTGDADSVPD | |
| | PQPLGQPPAAPSGLGTNTMATGSGAPMADNNEGADGVGNSSGNWHC | |
| | DSTWMGDRVITTSTRTWALPTYNNHLYKQISSQSGASNDNHYFGYS | |
| | TPWGYFDFNRFHCHFSPRDWQRLINNNWGFRPKRLNFKLFNIQVKE | |
| | VTQNDGTTTIANNLTSTVQVFTDSEYQLPYVLGSAHQGCLPPFPAD | |
| | VFMVPQYGYLTLNNGSQAVGRSSFYCLEYFPSQMLRTGNNFTFSYT | |
| | FEDVPFHSSYAHSQSLDRLMNPLIDQYLYYLSRTNTPSGTTTQSRL | |
| | QFSQAGASDIRDQSRNWLPGPCYRQQRVSKTSADNNNSEYSWTGAT | |
| | KYHLNGRDSLVNPGPAMASHKDDEEKFFPQSGVLIFGKQGSEKTNV | |
| | DIEKVMITDEEEIRTTNPVATEQYGSVSTNLQRGNRQAATADVNTQ | |
| | GVLPGMVWQDRDVYLQGPIWAKIPHTDGHFHPSPLMGGFGLKHPPP | |
| | QILIKNTPVPANPSTTFSAAKFASFITQYSTGQVSVEIEWELQKEN | |
| | SKRWNPEIQYTSNYNKSVNVDFTVDTNGVYSEPRPIGTRYLTRNL | |
| 3 | MAADGYLPDWLEDNLSEGIREWWALKPGVPQPKANQQHQDNRRGLV | AAV3B |
| | LPGYKYLGPGNGLDKGEPVNEADAAALEHDKAYDQQLKAGDNPYLK | |
| | YNHADAEFQERLQEDTSFGGNLGRAVFQAKKRILEPLGLVEEAAKT | |
| | APGKKRPVDQSPQEPDSSSGVGKSGKQPARKRLNFGQTGDSESVPD | |
| | PQPLGEPPAAPTSLGSNTMASGGGAPMADNNEGADGVGNSSGNWHC | |
| | DSQWLGDRVITTSTRTWALPTYNNHLYKQISSQSGASNDNHYFGYS | |
| | TPWGYFDFNRFHCHFSPRDWQRLINNNWGFRPKKLSFKLFNIQVKE | |
| | VTQNDGTTTIANNLTSTVQVFTDSEYQLPYVLGSAHQGCLPPFPAD | |
| | VFMVPQYGYLTLNNGSQAVGRSSFYCLEYFPSQMLRTGNNFQFSYT | |
| | FEDVPFHSSYAHSQSLDRLMNPLIDQYLYYLNRTQGTTSGTTNQSR | |
| | LLFSQAGPQSMSLQARNWLPGPCYRQQRLSKTANDNNNSNFPWTAA | |
| | SKYHLNGRDSLVNPGPAMASHKDDEEKFFPMHGNLIFGKEGTTASN | |
| | AELDNVMITDEEEIRTTNPVATEQYGTVANNLQSSNTAPTTRTVND | |
| | QGALPGMVWQDRDVYLQGPIWAKIPHTDGHFHPSPLMGGFGLKHPP | |
| | PQIMIKNTPVPANPPTTFSPAKFASFITQYSTGQVSVEIEWELQKE | |
| | NSKRWNPEIQYTSNYNKSVNVDFTVDTNGVYSEPRPIGTRYLTRNL | |
| 4 | MTDGYLPDWLEDNLSEGVREWWALQPGAPKPKANQQHQDNARGLVL | AAV4 |
| | PGYKYLGPGNGLDKGEPVNAADAAALEHDKAYDQQLKAGDNPYLKY | |
| | NHADAEFQQRLQGDTSFGGNLGRAVFQAKKRVLEPLGLVEQAGETA | |
| | PGKKRPLIESPQQPDSSTGIGKKGKQPAKKKLVFEDETGAGDGPPE | |
| | GSTSGAMSDDSEMRAAAGGAAVEGGQGADGVGNASGDWHCDSTWSE | |
| | GHVTTTSTRTWVLPTYNNHLYKRLGESLQSNTYNGFSTPWGYFDFN | |
| | RFHCHFSPRDWQRLINNNWGMRPKAMRVKIFNIQVKEVTTSNGETT | |
| | VANNLTSTVQIFADSSYELPYVMDAGQEGSLPPFPNDVFMVPQYGY | |
| | CGLVTGNTSQQQTDRNAFYCLEYFPSQMLRTGNNFEITYSFEKVPF | |
| | HSMYAHSQSLDRLMNPLIDQYLWGLQSTTTGTTLNAGTATTNFTKL | |
| | RPTNFSNFKKNWLPGPSIKQQGFSKTANQNYKIPATGSDSLIKYET | |
| | HSTLDGRWSALTPGPPMATAGPADSKFSNSQLIFAGPKQNGNTATV | |
| | PGTLIFTSEEELAATNATDTDMWGNLPGGDQSNSNLPTVDRLTALG | |
| | AVPGMVWQNRDIYYQGPIWAKIPHTDGHFHPSPLIGGFGLKHPPPQ | |
| | IFIKNTPVPANPATTFSSTPVNSFITQYSTGQVSVQIDWEIQKERS | |
| | KRWNPEVQFTSNYGQQNSLLWAPDAAGKYTEPRAIGTRYLTHHL | |
| 5 | MSFVDHPPDWLEEVGEGLREFLGLEAGPPKPKPNQQHQDQARGLVL | AAV5 |
| | PGYNYLGPGNGLDRGEPVNRADEVAREHDISYNEQLEAGDNPYLKY | |
| | NHADAEFQEKLADDTSFGGNLGKAVFQAKKRVLEPFGLVEEGAKTA | |
| | PTGKRIDDHFPKRKKARTEEDSKPSTSSDAEAGPSGSQQLQIPAQP | |
| | ASSLGADTMSAGGGGPLGDNNQGADGVGNASGDWHCDSTWMGDRVV | |
| | TKSTRTWVLPSYNNHQYREIKSGSVDGSNANAYFGYSTPWGYFDFN | |
| | RFHSHWSPRDWQRLINNYWGFRPRSLRVKIFNIQVKEVTVQDSTTT | |
| | IANNLTSTVQVFTDDDYQLPYVVGNGTEGCLPAFPPQVFTLPQYGY | |
| | ATLNRDNTENPTERSSFFCLEYFPSKMLRTGNNFEFTYNFEEVPFH | |
| | SSFAPSQNLFKLANPLVDQYLYRFVSTNNTGGVQFNKNLAGRYANT | |
| | YKNWFPGPMGRTQGWNLGSGVNRASVSAFATTNRMELEGASYQVPP | |
| | QPNGMTNNLQGSNTYALENTMIFNSQPANPGTTATYLEGNMLITSE | |
| | SETQPVNRVAYNVGGQMATNNQSSTTAPATGTYNLQEIVPGSVWME | |
| | RDVYLQGPIWAKIPETGAHFHPSPAMGGFGLKHPPPMMLIKNTPVP | |
| | GNITSFSDVPVSSFITQYSTGQVTVEMEWELKKENSKRWNPEIQYT | |
| | NNYNDPQFVDFAPDSTGEYRTTRPIGTRYLTRPL | |
| 6 | MAADGYLPDWLEDNLSEGIREWWDLKPGAPKPKANQQKQDDGRGLV | AAV6 |
| | LPGYKYLGPFNGLDKGEPVNAADAAALEHDKAYDQQLKAGDNPYLR | |
| | YNHADAEFQERLQEDTSFGGNLGRAVFQAKKRVLEPFGLVEEGAKT | |
| | APGKKRPVEQSPQEPDSSSGIGKTGQQPAKKRLNFGQTGDSESVPD | |
| | PQPLGEPPATPAAVGPTTMASGGGAPMADNNEGADGVGNASGNWHC | |
| | DSTWLGDRVITTSTRTWALPTYNNHLYKQISSASTGASNDNHYFGY | |
| | STPWGYFDFNRFHCHFSPRDWQRLINNNWGFRPKRLNFKLFNIQVK | |
| | EVTTNDGVTTIANNLTSTVQVFSDSEYQLPYVLGSAHQGCLPPFPA | |
| | DVFMIPQYGYLTLNNGSQAVGRSSFYCLEYFPSQMLRTGNNFTFSY | |
| | TFEDVPFHSSYAHSQSLDRLMNPLIDQYLYYLNRTQNQSGSAQNKD | |
| | LLFSRGSPAGMSVQPKNWLPGPCYRQQRVSKTKTDNNNSNFTWTGA | |
| | SKYNLNGRESIINPGTAMASHKDDKDKFFPMSGVMIFGKESAGASN | |
| | TALDNVMITDEEEIKATNPVATERFGTVAVNLQSSSTDPATGDVHV | |
| | MGALPGMVWQDRDVYLQGPIWAKIPHTDGHFHPSPLMGGFGLKHPP | |
| | PQILIKNTPVPANPPAEFSATKFASFITQYSTGQVSVEIEWELQKE | |
| | NSKRWNPEVQYTSNYAKSANVDFTVDNNGLYTEPRPIGTRYLTRPL | |
| 7 | MAADGYLPDWLEDNLSEGIREWWDLKPGAPKPKANQQKQDNGRGLV | AAV7 |
| | LPGYKYLGPFNGLDKGEPVNAADAAALEHDKAYDQQLKAGDNPYLR | |
| | YNHADAEFQERLQEDTSFGGNLGRAVFQAKKRVLEPLGLVEEGAKT | |
| | APAKKRPVEPSPQRSPDSSTGIGKKGQQPARKRLNFGQTGDSESVP | |
| | DPQPLGEPPAAPSSVGSGTVAAGGGAPMADNNEGADGVGNASGNWH | |
| | CDSTWLGDRVITTSTRTWALPTYNNHLYKQISSETAGSTNDNTYFG | |
| | YSTPWGYFDFNRFHCHFSPRDWQRLINNNWGFRPKKLRFKLFNIQV | |
| | KEVTTNDGVTTIANNLTSTIQVFSDSEYQLPYVLGSAHQGCLPPFP | |
| | ADVFMIPQYGYLTLNNGSQSVGRSSFYCLEYFPSQMLRTGNNFEFS | |
| | YSFEDVPFHSSYAHSQSLDRLMNPLIDQYLYYLARTQSNPGGTAGN | |
| | RELQFYQGGPSTMAEQAKNWLPGPCFRQQRVSKTLDQNNNSNFAWT | |
| | GATKYHLNGRNSLVNPGVAMATHKDDEDRFFPSSGVLIFGKTGATN | |
| | KTTLENVLMTNEEEIRPTNPVATEEYGIVSSNLQAANTAAQTQVVN | |
| | NQGALPGMVWQNRDVYLQGPIWAKIPHTDGNFHPSPLMGGFGLKHP | |
| | PPQILIKNTPVPANPPEVFTPAKFASFITQYSTGQVSVEIEWELQK | |
| | ENSKRWNPEIQYTSNFEKQTGVDFAVDSQGVYSEPRPIGTRYLTRN | |
| | L | |
| 8 | MAADGYLPDWLEDNLSEGIREWWALKPGAPKPKANQQKQDDGRGLV | AAV8 |
| | LPGYKYLGPFNGLDKGEPVNAADAAALEHDKAYDQQLQAGDNPYLR | |
| | YNHADAEFQERLQEDTSFGGNLGRAVFQAKKRVLEPLGLVEEGAKT | |
| | APGKKRPVEPSPQRSPDSSTGIGKKGQQPARKRLNFGQTGDSESVP | |
| | DPQPLGEPPAAPSGVGPNTMAAGGGAPMADNNEGADGVGSSSGNWH | |
| | CDSTWLGDRVITTSTRTWALPTYNNHLYKQISNGTSGGATNDNTYF | |
| | GYSTPWGYFDFNRFHCHFSPRDWQRLINNNWGFRPKRLSFKLFNIQ | |
| | VKEVTQNEGTKTIANNLTSTIQVFTDSEYQLPYVLGSAHQGCLPPF | |
| | PADVFMIPQYGYLTLNNGSQAVGRSSFYCLEYFPSQMLRTGNNFQF | |
| | TYTFEDVPFHSSYAHSQSLDRLMNPLIDQYLYYLSRTQTTGGTANT | |
| | QTLGFSQGGPNTMANQAKNWLPGPCYRQQRVSTTTGQNNNSNFAWT | |
| | AGTKYHLNGRNSLANPGIAMATHKDDEERFFPSNGILIFGKQNAAR | |
| | DNADYSDVMLTSEEEIKTTNPVATEEYGIVADNLQQQNTAPQIGTV | |
| | NSQGALPGMVWQNRDVYLQGPIWAKIPHTDGNFHPSPLMGGFGLKH | |
| | PPPQILIKNTPVPADPPTTFNQSKLNSFITQYSTGQVSVEIEWELQ | |
| | KENSKRWNPEIQYTSNYYKSTSVDFAVNTEGVYSEPRPIGTRYLTR | |
| | NL | |
| 9 | MAADGYLPDWLEDNLSEGIREWWALKPGAPQPKANQQHQDNARGLV | AAV9 |
| | LPGYKYLGPGNGLDKGEPVNAADAAALEHDKAYDQQLKAGDNPYLK | |
| | YNHADAEFQERLKEDTSFGGNLGRAVFQAKKRLLEPLGLVEEAAKT | |
| | APGKKRPVEQSPQEPDSSAGIGKSGAQPAKKRLNFGQTGDTESVPD | |
| | PQPIGEPPAAPSGVGSLTMASGGGAPVADNNEGADGVGSSSGNWHC | |
| | DSQWLGDRVITTSTRTWALPTYNNHLYKQISNSTSGGSSNDNAYFG | |
| | YSTPWGYFDFNRFHCHFSPRDWQRLINNNWGFRPKRLNFKLFNIQV | |
| | KEVTDNNGVKTIANNLTSTVQVFTDSDYQLPYVLGSAHEGCLPPFP | |
| | ADVFMIPQYGYLTLNDGSQAVGRSSFYCLEYFPSQMLRTGNNFQFS | |
| | YEFENVPFHSSYAHSQSLDRLMNPLIDQYLYYLSKTINGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQSAQAQAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKE | |
| | NSKRWNPEIQYTSNYYKSNNVEFAVNTEGVYSEPRPIGTRYLTRNL | |
| 10 | MAADGYLPDWLEDNLSEGIREWWDLKPGAPKPKANQQKQDDGRGLV | AAV10 |
| | LPGYKYLGPFNGLDKGEPVNAADAAALEHDKAYDQQLKAGDNPYLR | |
| | YNHADAEFQERLQEDTSFGGNLGRAVFQAKKRVLEPLGLVEEAAKT | |
| | APGKKRPVEPSPQRSPDSSTGIGKKGQQPAKKRLNFGQTGESESVP | |
| | DPQPIGEPPAGPSGLGSGTMAAGGGAPMADNNEGADGVGSSSGNWH | |
| | CDSTWLGDRVITTSTRTWALPTYNNHLYKQISNGTSGGSTNDNTYF | |
| | GYSTPWGYFDFNRFHCHFSPRDWQRLINNNWGFRPKRLSFKLFNIQ | |
| | VKEVTQNEGTKTIANNLTSTIQVFTDSEYQLPYVLGSAHQGCLPPF | |
| | PADVFMIPQYGYLTLNNGSQAVGRSSFYCLEYFPSQMLRTGNNFEF | |
| | SYTFEDVPFHSSYAHSQSLDRLMNPLIDQYLYYLSRTQSTGGTQGT | |
| | QQLLFSQAGPANMSAQAKNWLPGPCYRQQRVSTTLSQNNNSNFAWT | |
| | GATKYHLNGRDSLVNPGVAMATHKDDEERFFPSSGVLMFGKQGAGR | |
| | DNVDYSSVMLTSEEEIKTTNPVATEQYGVVADNLQQANTGPIVGNV | |
| | NSQGALPGMVWQNRDVYLQGPIWAKIPHTDGNFHPSPLMGGFGLKH | |
| | PPPQILIKNTPVPADPPTTFSQAKLASFITQYSTGQVSVEIEWELQ | |
| | KENSKRWNPEIQYTSNYYKSTNVDFAVNTEGTYSEPRPIGTRYLTR | |
| | NL | |
| 11 | MAADGYLPDWLEDNLSEGIREWWDLKPGAPKPKANQQKQDDGRGLV | AAV11 |
| | LPGYKYLGPFNGLDKGEPVNAADAAALEHDKAYDQQLKAGDNPYLR | |
| | YNHADAEFQERLQEDTSFGGNLGRAVFQAKKRVLEPLGLVEEGAKT | |
| | APGKKRPLESPQEPDSSSGIGKKGKQPARKRLNFEEDTGAGDGPPE | |
| | GSDTSAMSSDIEMRAAPGGNAVDAGQGSDGVGNASGDWHCDSTWSE | |
| | GKVTTTSTRTWVLPTYNNHLYLRLGTTSSSNTYNGFSTPWGYFDFN | |
| | RFHCHFSPRDWQRLINNNWGLRPKAMRVKIFNIQVKEVTTSNGETT | |
| | VANNLTSTVQIFADSSYELPYVMDAGQEGSLPPFPNDVFMVPQYGY | |
| | CGIVTGENQNQTDRNAFYCLEYFPSQMLRTGNNFEMAYNFEKVPFH | |
| | SMYAHSQSLDRLMNPLLDQYLWHLQSTTSGETLNQGNAATTFGKIR | |
| | SGDFAFYRKNWLPGPCVKQQRFSKTASQNYKIPASGGNALLKYDTH | |
| | YTLNNRWSNIAPGPPMATAGPSDGDFSNAQLIFPGPSVTGNTTTSA | |
| | NNLLFTSEEEIAATNPRDTDMFGQIADNNQNATTAPITGNVTAMGV | |
| | LPGMVWQNRDIYYQGPIWAKIPHADGHFHPSPLIGGFGLKHPPPQI | |
| | FIKNTPVPANPATTFTAARVDSFITQYSTGQVAVQIEWEIEKERSK | |
| | RWNPEVQFTSNYGNQSSMLWAPDTTGKYTEPRVIGSRYLTNHL | |
| 12 | MAADGYLPDWLEDNLSEGIREWWALKPGAPQPKANQQHQDNGRGLV | AAV12 |
| | LPGYKYLGPFNGLDKGEPVNEADAAALEHDKAYDKQLEQGDNPYLK | |
| | YNHADAEFQQRLATDTSFGGNLGRAVFQAKKRILEPLGLVEEGVKT | |
| | APGKKRPLEKTPNRPTNPDSGKAPAKKKQKDGEPADSARRTLDFED | |
| | SGAGDGPPEGSSSGEMSHDAEMRAAPGGNAVEAGQGADGVGNASGD | |
| | WHCDSTWSEGRVTTTSTRTWVLPTYNNHLYLRIGTTANSNTYNGFS | |
| | TPWGYFDFNRFHCHFSPRDWQRLINNNWGLRPKSMRVKIFNIQVKE | |
| | VTTSNGETTVANNLTSTVQIFADSTYELPYVMDAGQEGSFPPFPND | |
| | VFMVPQYGYCGVVTGKNQNQTDRNAFYCLEYFPSQMLRTGNNFEVS | |
| | YQFEKVPFHSMYAHSQSLDRMMNPLLDQYLWHLQSTTTGNSLNQGT | |
| | ATTTYGKITTGDFAYYRKNWLPGACIKQQKFSKNANQNYKIPASGG | |
| | DALLKYDTHTTLNGRWSNMAPGPPMATAGAGDSDFSNSQLIFAGPN | |
| | PSGNTTTSSNNLLFTSEEEIATTNPRDTDMFGQIADNNQNATTAPH | |
| | IANLDAMGIVPGMVWQNRDIYYQGPIWAKVPHTDGHFHPSPLMGGF | |
| | GLKHPPPQIFIKNTPVPANPNTTFSAARINSFLTQYSTGQVAVQID | |
| | WEIQKEHSKRWNPEVQFTSNYGTQNSMLWAPDNAGNYHELRAIGSR | |
| | FLTHHL | |
| 13 | MTDGYLPDWLEDNLSEGVREWWALQPGAPKPKANQQHQDNARGLVL | AAV13 |
| | PGYKYLGPGNGLDKGEPVNAADAAALEHDKAYDQQLKAGDNPYLKY | |
| | NHADAEFQERLQEDTSFGGNLGRAVFQAKKRILEPLGLVEEAAKTA | |
| | PGKKRPVEQSPAEPDSSSGIGKSGQQPARKRLNFGQTGDTESVPDP | |
| | QPLGQPPAAPSGVGSTTMASGGGAPMADNNEGADGVGNSSGNWHCD | |
| | SQWLGDRVITTSTRTWALPTYNNHLYKQISSQSGATNDNHYFGYST | |
| | PWGYFDFNRFHCHFSPRDWQRLINNNWGFRPKRLNFKLFNIQVKEV | |
| | TQNDGTTTIANNLTSTVQVFTDSEYQLPYVLGSAHQGCLPPFPADV | |
| | FMVPQYGYLTLNNGSQAVGRSSFYCLEYFPSQMLRTGNNFQFSYTF | |
| | EDVPFHSSYAHSQSLDRLMNPLIDQYLYYLNRTQTASGTQQSRLLF | |
| | SQAGPTSMSLQAKNWLPGPCYRQQRLSKQANDNNNSNFPWTGATKY | |
| | HLNGRDSLVNPGPAMASHKDDKEKFFPMHGTLIFGKEGTNANNADL | |
| | ENVMITDEEEIRTTNPVATEQYGTVSNNLQNSNAGPTTGTVNHQGA | |
| | LPGMVWQDRDVYLQGPIWAKIPHTDGHFHPSPLMGGFGLKHPPPQI | |
| | MIKNTPVPANPPTNFSAAKFASFITQYSTGQVSVEIEWELQKENSK | |
| | RWNPEIQYTSNYNKSVNVDFTVDTNGVYSEPRPIGTRYLTRNL | |
| 14 | MAADGYLPDWLEDNLSEGIREWWALKPGAPQPKANQQHQDNARGLV | AAV9 |
| | LPGYKYLGPGNGLDKGEPVNAADAAALEHDKAYDQQLKAGDNPYLK | N272A |
| | YNHADAEFQERLKEDTSFGGNLGRAVFQAKKRLLEPLGLVEEAAKT | |
| | APGKKRPVEQSPQEPDSSAGIGKSGAQPAKKRLNFGQTGDTESVPD | |
| | PQPIGEPPAAPSGVGSLTMASGGGAPVADNNEGADGVGSSSGNWHC | |
| | DSQWLGDRVITTSTRTWALPTYNNHLYKQISNSTSGGSSNDAAYFG | |
| | YSTPWGYFDFNRFHCHFSPRDWQRLINNNWGFRPKRLNFKLFNIQV | |
| | KEVTDNNGVKTIANNLTSTVQVFTDSDYQLPYVLGSAHEGCLPPFP | |
| | ADVFMIPQYGYLTLNDGSQAVGRSSFYCLEYFPSQMLRTGNNFQFS | |
| | YEFENVPFHSSYAHSQSLDRLMNPLIDQYLYYLSKTINGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQSAQAQAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKE | |
| | NSKRWNPEIQYTSNYYKSNNVEFAVNTEGVYSEPRPIGTRYLTRNL | |
| 15 | QQNGTRPS | QQNGTRPS |
| 16 | MAADGYLPDWLEDNLSEGIREWWALKPGAPQPKANQQHQDNARGLV | CAP2 |
| | LPGYKYLGPGNGLDKGEPVNAADAAALEHDKAYDQQLKAGDNPYLK | |
| | YNHADAEFQERLKEDTSFGGNLGRAVFQAKKRLLEPLGLVEEAAKT | |
| | APGKKRPVEQSPQEPDSSAGIGKSGAQPAKKRLNFGQTGDTESVPD | |
| | PQPIGEPPAAPSGVGSLTMASGGGAPVADNNEGADGVGSSSGNWHC | |
| | DSQWLGDRVITTSTRTWALPTYNNHLYKQISNSTSGGSSNDAAYFG | |
| | YSTPWGYFDFNRFHCHFSPRDWQRLINNNWGFRPKRLNFKLFNIQV | |
| | KEVTDNNGVKTIANNLTSTVQVFTDSDYQLPYVLGSAHEGCLPPFP | |
| | ADVFMIPQYGYLTLNDGSQAVGRSSFYCLEYFPSQMLRTGNNFQFS | |
| | YEFENVPFHSSYAHSQSLDRLMNPLIDQYLYYLSKTINGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQSAGGGSQQNGTRP | |
| | SGGGGSAQAQTGWVQNQGILPGMVWQDRDVYLQGPIWAKIPHTDGN | |
| | FHPSPLMGGFGMKHPPPQILIKNTPVPADPPTAFNKDKLNSFITQY | |
| | STGQVSVEIEWELQKENSKRWNPEIQYTSNYYKSNNVEFAVNTEGV | |
| | YSEPRPIGTRYLTRNL | |

1. A method of delivering a nucleic acid molecule to a motor neuron in an individual, the method comprising:
administering a variant adeno-associated virus (AAV) comprising the nucleic acid molecule to the individual, wherein the variant AAV comprises a variant capsid protein comprising an amino acid insertion within loop VIII, wherein the amino acid insertion comprises SEQ ID NO: 15.

2. A method of delivering a therapeutic nucleic acid molecule to a motor neuron in a subject having a motor neuron disease, the method comprising:
administering a variant adeno-associated virus (AAV) comprising the therapeutic nucleic acid molecule to the individual, wherein the variant AAV comprises a variant capsid protein comprising an amino acid insertion within loop VIII, wherein the amino acid insertion comprises SEQ ID NO: 15.

3. The method of any one of claims 1-2, wherein the motor neuron is an upper motor neuron or a lower motor neuron.

4. The method of any one of claims 1-3, wherein the motor neuron is located within the frontal cortex in the brain.

5. The method of any one of claims 1-4, wherein the motor neuron is located within a spine.

6. The method of any one of claims 1-5, wherein the motor neuron is located within a ventral horn region of a spinal cord.

7. The method of any one of claims 1-6, wherein the motor neuron is located within a cervical region to a sacral region of a spine.

8. The method of any one of claims 1-7, wherein the motor neuron is located within a cervical region of a spine, a thoracic region of the spine, a lumbar region of the spine, or a sacral region of the spine.

9. A method of treating a motor neuron disease in an individual, comprising:
administering a variant adeno-associated virus (AAV) comprising a therapeutic nucleic acid molecule to the individual, wherein the variant AAV comprises a variant capsid protein comprising an amino acid insertion within loop VIII, wherein the amino acid insertion comprises SEQ ID NO: 15.

10. The method of claim 9, wherein the variant AAV delivers the therapeutic nucleic acid of to a motor neuron.

11. The method of claim 10, wherein the motor neuron is an upper motor neuron or a lower motor neuron.

12. The method of any one of claims 10-11, wherein the motor neuron is located within the frontal cortex in the brain.

13. The method of any one of claims 10-12, wherein the lower motor neuron is located within a ventral horn region of a spinal cord.

14. The method of any one of claims 10 and 12-13, wherein the motor neuron is located within a spine.

15. The method of any one of claims 10 and 12-14, wherein the motor neuron is located within a cervical region to a sacral region of a spine.

16. The method of any one of claims 10 and 12-15, wherein the motor neuron is located within a cervical region of a spine, a thoracic region of the spine, a lumbar region of the spine, or a sacral region of the spine.

17. A method of delivering a nucleic acid to a motor neuron, the method comprising:
contacting the motor neuron with a variant adeno-associated virus (AAV) comprising the nucleic acid molecule, wherein the variant AAV comprises a variant capsid protein comprising an amino acid insertion within loop VIII, wherein the amino acid insertion comprises SEQ ID NO: 15.

18. The method of any one of claims 1-17, wherein the variant capsid protein is an AAV serotype 9 (AAV9) capsid protein.

19. The method of claim 18, wherein the AAV9 capsid protein comprises an N272A mutation.

20. The method of any one of claims 1-19, wherein the variant capsid protein without the amino acid insertion comprises 90% sequence identity to SEQ ID NO: 9.

21. The method of any one of claims 1-19, wherein the variant capsid protein without the amino acid insertion comprises 90% sequence identity to SEQ ID NO: 14.

22. The method of any one of claims 1-21, wherein the amino acid insertion further comprises a linker.

23. The method of any one of claims 1-22, wherein the amino acid insertion further comprises the formula: 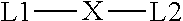
wherein:
L1 comprises a first amino acid linker sequence;
X comprises SEQ ID NO: 15; and
L2 is a second linker sequence.

24. The method of claim 23, wherein L1 and L2 are a different sequence.

25. The method of claim 23, wherein L1 and L2 are an identical sequence.

26. The method of any one of claims 1 to 25, wherein the motor neuron is choline acetyltransferase positive.

27. The method of any one of claims 1 to 26, wherein the motor neuron is SMI-32 positive.

28. The method of any one of claims 1 to 27, wherein the variant AAV exhibits a higher transduction of choline acetyltransferase-positive motor neurons when compared to transduction attainable by an equivalent dose of a control AAV (e.g., AAV9) comprising a wild-type AAV9 capsid protein (SEQ ID NO: 9).

29. The method of any one of claims 1 to 28, wherein the variant AAV exhibits reduced transduction of choline acetyltransferase-negative neurons and non-neuronal cells (e.g., glia) when compared to a control AAV (e.g., AAV9) comprising a wild-type AAV9 capsid protein (SEQ ID NO: 9).

30. The method of any one of claims 1 to 29, wherein the variant AAV exhibits increased transduction of upper motor neurons when compared to transduction attainable by an equivalent dose of a control AAV (e.g., AAV9) comprising a wild-type AAV9 capsid protein (SEQ ID NO: 9).

31. The method of any one of claims 1 to 30, wherein the variant AAV exhibits reduced transduction of non-neuronal cells (e.g., glia), when compared to a control AAV (e.g., AAV9) comprising a wild-type AAV9 capsid protein (SEQ ID NO: 9).

32. The method of any one of claims 1 to 31, wherein the variant AAV transduces a higher ratio of choline acetyltransferase-positive motor neurons to choline acetyltransferase-negative neurons and non-neuronal cells (e.g., glia) when compared to a control AAV (e.g., AAV9) comprising a wild-type AAV9 capsid protein (SEQ ID NO: 9).

33. The method of any one of claims 1 to 32, wherein the variant AAV transduces a higher ratio of upper motor neurons to other types of neurons and non-neuronal cells (e.g., glia) when compared to a control AAV (e.g., AAV9) comprising a wild-type AAV9 capsid protein (SEQ ID NO: 9).

34. The method of any one of claims 1 to 33, wherein the variant capsid comprises SEQ ID NO: 15 and has at least 85%, 90%, 95%, 97%, 98%, 99%, or 100% sequence identity to SEQ ID NO: 16.
